(19) United States
(12) Patent Application Publication
(10) Pub. No.: US 2023/0181928 A1
(43) Pub. Date: Jun. 15, 2023

(54) BOLUS, BOLUS POSITIONING SYSTEM AND METHOD OF MANUFACTURING THE SAME

(71) Applicant: GENESISCARE VENTURES PTY LTD, Alexandria (AU)

(72) Inventors: Ian QUIN, Alexandria (AU); Jacob GRIFFITHS, Alexandria (AU); Raj PARAMANATHAN, Alexandria (AU); Gordon WALLACE, Alexandria (AU); Daniel LAWRENCE, Alexandria (AU); Stephen BEIRNE, Alexandria (AU); Simon BURRAGE, Alexandria (AU)

(21) Appl. No.: 18/165,914

(22) Filed: Feb. 7, 2023

Related U.S. Application Data
(63) Continuation of application No. PCT/AU2022/050512, filed on May 26, 2022.

(30) Foreign Application Priority Data
May 26, 2021 (AU) 2021901578

(51) Int. Cl. A61N 5/10

(52) U.S. Cl. CPC A61N 5/103; A61N 5/1075

(57) ABSTRACT

In one form, a bolus is configured to fit over a target body position and includes an internal relatively rigid endoskeleton structure and a surrounding relatively non-rigid skin interfacing layer. A positioning and locking system may include an indexing plate mountable to a fixture. The indexing plate includes a plurality of multiposition-enabling formations and corresponding reference locations and is configured to engage with at least one locking mechanism. The at least one locking mechanism is movable between and lockable relative to the multiposition-enabling formations. The locking mechanism is also configured to interface between the indexing plate and accessories/devices that require immobilisation, accurate and/or repeatable positioning, e.g. the bolus. In one example, an interconnecting formation is provided for rigidly interconnecting the at least one locking mechanism and the bolus in a recordable position for enabling repeated radiotherapy treatments on the same target body portion of the user in the same position.

### FIELD OF THE INVENTION

The present invention relates to a bolus for radiotherapy, a bolus positioning system and to a method of manufacturing the same. In particular, the present invention relates to a bolus and bolus positioning system for facilitating accurate and repeatable positioning of a target body portion of a patient undergoing repeated sessions of radiotherapy treatment.

### BACKGROUND OF THE INVENTION

During radiotherapy, high intensity ionising radiation is used to destroy cancerous cells in a targeted affected area on a patient. Precise targeting is essential to ensure that cancerous cells are destroyed rather than the healthy surrounding tissue. This requires the target body portion of the patient to be immobilised and to be accurately positioned over multiple treatment sessions. This is a laborious and time-consuming process involving repeated CT scanning for each session and subsequent immobilisation typically involving the use of a vacuum bag prior to scanning.

Radiation beams generally deposit a maximum dose below rather than on the skin surface. In the case where a surface dose is required for skin cancers, non-melanoma skin cancer (NMSC) and the like, a tissue equivalent material known as a bolus is placed on the affected area to control the depth which a therapeutic dose of radiation is deposited and ensure that the radiation peak is at or close to the affected area of the skin. The thickness of the bolus may be varied to ensure the correct level of radiation is applied at the correct depth. Boluses are typically manufactured manually by radiation therapists using wax or a thermoplastic material. This is a time-consuming and generally inaccurate process. In addition, in the case of repeat therapies, both the bolus and the affected area needs to be accurately and precisely re-positioned.

Reference to any prior art in the specification is not, and should not be taken as, an acknowledgment or any form of suggestion that this prior art forms part of the common general knowledge in any jurisdiction or that this prior art could reasonably be expected to be understood, regarded as relevant and/or combined with other pieces of prior art by a person skilled in the art.

### SUMMARY OF THE INVENTION

According to a first aspect of the invention there is provided a bolus assembly for facilitating radiotherapy on a target body portion of a user, the bolus assembly comprising: a customised bolus for fitting over the target body portion, the customised bolus comprising an internal relatively rigid endoskeleton structure and a surrounding relatively non-rigid skin interfacing layer; an indexing plate mountable to a fixture and including a plurality of multiposition-enabling formations and corresponding reference locations; at least one locking mechanism movable between and lockable relative to the multiposition-enabling formations; at least one interconnecting formation for rigidly interconnecting the at least one locking mechanism and the customised bolus in a recordable position for enabling repeated radiotherapy treatments on the same target body portion of the user in the same position.

In some embodiments, the multi-position-enabling formations may comprise a plurality of tracks or channels within which the at least one locking mechanism is movable. The at least one locking mechanism may include a locking plate and at least one clamping assembly movable between a clamping or locking position in which it is immobilised relative to the tracks or channels and a released position in which it is free to move along the tracks or channels. In the released position the locking mechanism may be free to move both linearly along the tracks or channels and to rotate angularly relative to the tracks or channels to facilitate positioning of the bolus prior to locking. The angular rotation or yawing of the at least one locking mechanism may be limited to up to approximately 45 degrees of movement in each direction. The recordable position is viewable through a viewing window provided on the locking plate. The viewing window may be configured as a through-hole to receive a locking or aligning pin for aligning the locking plate with a corresponding indexing recess in the indexing plate.

In some embodiments, the at least one interconnecting formation may include a bolus connector extending from the endoskeleton structure, a complemental connector defined in the locking mechanism, and a lock formation for rigidly interlocking the bolus and complemental connector. The connector and complemental connector may include at least one respective substantially V-shaped projection and at least one corresponding substantially V-shaped recess.

In some embodiments, the reference locations may have associated indexing indicia for enabling the position of the customised bolus to be recorded for a particular user.

In some embodiments, the bolus assembly includes a cradle component for initially locating a target body portion of the user, the cradle component being interchangeable with the customised bolus and being configured to locate and support a vacuum bag for immobilising the body portion prior to 3D scanning of the body portion. The cradle component is radio translucent, and includes a cradle portion and at least one cradle connector extending from the cradle portion for interlocking with the complemental connector defined in the locking mechanism, and a lock formation for rigidly interlocking the cradle and complemental connector. The at least one cradle connector and complemental connector may include at least one respective substantially V-shaped projection and at least one corresponding substantially V-shaped recess. The cradle connector may be anchored in position to the cradle portion via a backing plate positioned substantially centrally on the base of the cradle portion. At least one support sled may be fixed to the base of the cradle component for providing additional load bearing support for the cradle component by resting on an upper surface of the indexing plate.

In some embodiments, the surrounding relatively non-rigid skin interfacing layer may be overmoulded over the internal relatively rigid endoskeleton structure. A relatively rigid shell defining a moulding cavity and the endoskeleton structure located within the cavity may be formed by an additive manufacturing process such as 3D printing. The shell may be formed with at least one injection port for injecting curable moulding material into the moulding cavity to form the relatively non-rigid skin interfacing layer.

In some embodiments, the relatively non-rigid skin interfacing layer of the customised bolus may be provided with a longitudinally extending cut providing a clamshell-type opening to facilitate the bolus to be opened and snugly fitted in place around the target portion of the user.

In some embodiments, the relatively non-rigid skin interfacing layer may be formed with material having a shore A hardness between 10 and 60.

In some embodiments, the relatively non-rigid skin interfacing layer may be formed from a material selected from a group including at least one of silicone, polyurethane, Superflab, thermoplastic-waxes and latex rubbers.

In some embodiments, the internal relatively rigid endoskeleton structure may be extended so as to substantially wrap around at least the base area of the customised bolus.

In some embodiments, the indexing plate may include at least one set of cross hairs for facilitating positioning the target body portion of the user with the customised bolus and the indexing plate without the need for user tattoo markers for positioning.

In some embodiments, the multiposition-enabling formations further comprise a plurality of locating pin holes for locating the at least one locking mechanism. The at least one locking mechanism may include at least one locating pin for locating the at least one locking mechanism at a desired locating pin hole of the plurality of locating pin holes.

In some embodiments, the at least one locking mechanism includes a locking pin extending through at least one aperture in the complemental connector; the at least one interconnecting formation includes at least one aperture; and in a locking state, the locking pin is inserted through aligned apertures of the complemental connector and the at least one interconnecting formation.

In some embodiments, the at least one locking mechanism includes a cam lock; and the at least one interconnecting formation includes at least one slot to receive the cam lock in a locking state.

According to a second aspect of the invention there is provided a method of forming an overmoulded customised bolus for radiation therapy comprising: immobilising a body portion of a user in a fixed position; recording the fixed position using position referencing means; acquiring user specific data from a 3D scan of the body portion of the user, including a target body portion; processing the data obtained from the 3D scan; generating a bolus file including bolus thickness data; generating an STL file from the bolus file including a sacrificial moulding shell and an endoskeleton structure; combining the STL file with an interconnect STL file; exporting the combined STL file to a 3D printing format; 3D printing the sacrificial moulding shell and the endoskeleton structure; overmoulding the endoskeleton structure by filling the sacrificial shell with curable material; and removing the sacrificial shell.

In some embodiments, acquiring the user specific data may comprise: placing a vacuum bag around the body portion of the user, including a target body portion; placing the body portion of the user in a cradle component that is attached to a indexing plate; immobilising the body portion of the user within the vacuum bag in the cradle component; adjusting the position of the cradle component on the indexing plate; locking the cradle component in place on the indexing plate; and performing the 3D scan. The cradle component may include at least one cradle connector and the position of the at least one cradle connector relative to the body portion of the user is recorded in the locked position.

In some embodiments, processing the data obtained from the 3D scan may comprise: identifying the skin boundary of the body portion of the user; identifying the relative location of the at least one cradle connector; and identifying the desired radiation treatment area.

In some embodiments, the sacrificial moulding shell comprises outer and inner walls defining a moulding cavity representative of the bolus thickness.

In some embodiments, filling the sacrificial shell with curable material may comprise: connecting at least one syringe into a corresponding injection port in the sacrificial shell, filling the at least one syringe with the curable material, and injecting the curable material into the sacrificial shell.

In some embodiments, the thickness of the sacrificial moulding shell may be from 0.7mm to 1.5 mm.

In some embodiments, the endoskeleton structure may be formed from a relatively hard polymer such as Polylactic Acid (PLA) and Acrylonitrile Butadiene Styrene (ABS).

According to a third aspect of the invention there is provided a customised bolus for fitting over a target body portion of a user, the customised bolus comprising: an internal relatively rigid endoskeleton structure; a relatively non-rigid skin interfacing layer surrounding the internal relatively rigid endoskeleton structure; and at least one connector extending from the endoskeleton structure, the connector being rigidly interconnectable to a locking mechanism which is detachably connectable to an indexing plate for enabling repeated radiotherapy treatments on the same target body portion of the user.

In some embodiments, the relatively non-rigid skin interfacing layer may be overmoulded over the internal relatively rigid endoskeleton structure. A relatively rigid shell defining a moulding cavity and the endoskeleton structure located within the cavity may be formed by an additive manufacturing process such as 3D printing. The shell may formed with at least one injection port for injecting curable moulding material into the moulding cavity to form the relatively non-rigid skin interfacing layer.

In some embodiments, the relatively non-rigid skin interfacing layer of the customised bolus may be provided with a longitudinally extending cut providing a clamshell-type opening to facilitate the customised bolus to be opened and snugly fitted in place around the target portion of the user.

In some embodiments, the relatively non-rigid skin interfacing layer may be formed with material having a shore A hardness between 10 and 60.

In some embodiments, the relatively non-rigid skin interfacing layer may be formed from a material selected from a group selected from at least one of silicone, polyurethane, Superflab, thermoplastic-waxes and latex rubbers.

In some embodiments, the internal relatively rigid endoskeleton structure may be extended so as to substantially wrap around at least the base area of the customised bolus.

In some embodiments, the at least one connector may be integrally formed with the endoskeleton structure.

In some embodiments, the endoskeleton structure may be formed with one or more reinforcing ribs.

According to a fourth aspect of the invention there is provided a locking assembly for locking a cradle component for initially locating a target body portion of the user during 3D scanning or a bolus during radiotherapy on the target body portion of the user, the locking assembly comprising: an indexing plate mountable to a fixture and including a plurality of multiposition-enabling formations and corresponding reference locations; at least one locking mechanism movable between and lockable relative to the multiposition-enabling formations; and at least one interconnecting formation for rigidly interconnecting the at least one locking mechanism and the bolus in a recordable position for enabling repeated radiotherapy treatments on the same target body portion of the user in the same position.

In some embodiments, the multiposition-enabling formations may comprise a plurality of tracks or channels within which the at least one locking mechanism is movable. The at least one locking mechanism may include a locking plate and at least one clamping assembly movable between a clamping or locking position in which it is immobilised relative to the tracks or channels and a released position in which it is free to move along the tracks or channels. In the released position the locking mechanism may be free to move both linearly along the tracks or channels and to rotate angularly relative to the tracks or channels to facilitate positioning of the bolus prior to locking. The angular rotation or yawing of the at least one locking mechanism may be limited to up to approximately 45 degrees of movement in each direction. The recordable position may be viewable through a viewing window provided on the locking plate. The viewing window may be configured as a through-hole to receive a locking or aligning pin for aligning the locking plate with a corresponding indexing recess in the indexing plate.

In some embodiments, the at least one interconnecting formation may include a connector defined in the locking mechanism, and a lock formation for rigidly interlocking the bolus and connector.

In some embodiments, the reference locations may have associated indexing indicia for enabling the position of the bolus to be recorded for a particular user.

In some embodiments, the indexing plate may include at least one set of cross hairs for facilitating positioning the target body portion of the user with the customised bolus and the indexing plate without the need for user tattoo markers for positioning.

Further aspects of the present invention and further embodiments of the aspects described in the preceding paragraphs will become apparent from the following description, given by way of example and with reference to the accompanying drawings.

As used herein, except where the context requires otherwise, the term “comprise” and variations of the term, such as “comprising”, “comprises” and “comprised”, are not intended to exclude further additions, components, integers or steps.

### BRIEF DESCRIPTION OF THE DRAWINGS

FIG. 1A illustrates a top plan view of a first embodiment of assembled components of part of a bolus positioning system including a C-channel component and an interconnected indexing plate;

FIG. 1B illustrates a front view of the assembled components of FIG. 1A;

FIG. 1C illustrates a side view of the assembled components of FIG. 1A;

FIG. 1D illustrates a perspective view of the assembled components of FIG. 1A;

FIG. 1E illustrates a top plan view of a second embodiment of assembled components of part of a bolus positioning system including a C-channel component and an interconnected indexing plate;

FIG. 1F illustrates a front view of the assembled components of FIG. 1E;

FIG. 1G illustrates a side view of the assembled components of FIG. 1E;

FIG. 1H illustrates a perspective view of the assembled components of FIG. 1E;

FIG. 2A illustrates a top plan view of a first embodiment of assembled components of a part of a bolus positioning system including a bolus component and the interconnected indexing plate;

FIG. 2B illustrates a front view of the assembled components of FIG. 2A;

FIG. 2C illustrates a perspective view of the assembled components of FIG. 2A;

FIG. 3A illustrates a perspective view of a C-channel component of the bolus assembly of FIGS. 1A to 1D;

FIG. 3B illustrates a side view of the C-channel component of FIG. 3A;

FIG. 3C illustrates an underplan view of the C-channel component of FIG. 3A;

FIG. 3D illustrates an end-on view of the C-channel component of FIG. 3A;

FIG. 3E illustrates a perspective view of a C-channel component of the bolus assembly of FIGS. 1E to 1H;

FIG. 3F illustrates a side view of the C-channel component of FIG. 3E;

FIG. 3G illustrates an underplan view of the C-channel component of FIG. 3E;

FIG. 3H illustrates an end-on view of the C-channel component of FIG. 3E;

FIGS. 4A to 4C illustrate respective top, side and end-on views of an embodiment of a bolus component of the bolus assembly of FIG. 2A to 2C;

FIG. 4D shows a partly cutaway perspective view of the bolus component of FIGS. 4A to 4C;

FIGS. 4E and 4F illustrate respective cross-sectional and detailed cross-sectional views of bolus component of FIG. 4D;

FIG. 4G shows a more detailed perspective view of an endoskeleton structure of the bolus component of FIGS. 4E and 4F;

FIG. 4H shows a cross-sectional view of another embodiment of the bolus embodiment with an endoskeleton structure extended so as to substantially wrap around the base area of the bolus component;

FIG. 4I shows a perspective view of an endoskeleton structure of the bolus component with through-holes formed with the bolus interconnectors being initially printed in a diamond or square shape;

FIGS. 5A, 5B and 5C illustrate respective perspective, top plan and detailed sectional views of a further embodiment of a bolus component;

FIGS. 6A and 6B illustrate respective side and detailed views of a manufacturing step of the bolus component of FIG. 5A;

FIG. 6C shows a perspective view of a completed bolus component;

FIGS. 7A to 7E illustrate respective perspective, top plan, front, perspective exploded and perspective cross sectional views of an exemplary locking plate forming part of the bolus system;

FIG. 7F illustrates a top plan view of an angular movement configuration example of a locking plate forming part of the bolus system;

FIGS. 8A and 8B illustrate respective perspective and detailed top plan views of the locking plate clamped onto the indexing plate;

FIG. 9 illustrates a flow diagram of the steps involved in manufacturing and positioning the bolus component; and

FIG. 10 illustrates a flow diagram of the steps involved in operating the bolus assembly.

FIGS. 11A-11E illustrate an example of CT scanning results of a user's right forearm positioned in a customised bolus component;

FIGS. 12A and 12B illustrate respective perspective and top plan views of another exemplary indexing plate;

FIGS. 13A to 13D illustrate respective perspective, top plan, front and underplan views of another exemplary locking plate;

FIG. 13E illustrates an unplugged configuration of a locating pin assembly of the locking plate in FIGS. 13A to 13D.

FIGS. 14A and 14B illustrate respective perspective and top plan views of the indexing plate in FIG. 12 and the locking plate in FIGS. 13A to 13D.

FIGS. 15A-15E illustrate respective perspective, first side, second side, underplan and end-on views of another exemplary C-channel component.

FIGS. 16A-16G illustrate respective perspective, first side, detailed first side, second side, underplan, end-on and detailed end-on views of another exemplary bolus component.

### DETAILED DESCRIPTION OF EMBODIMENTS

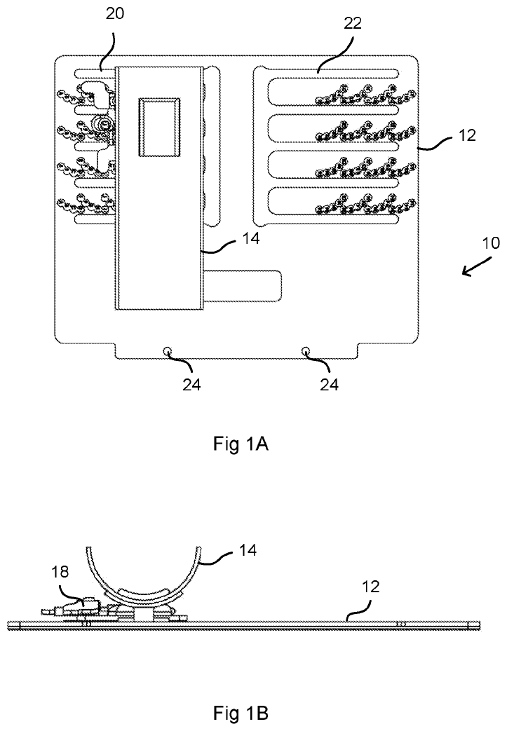

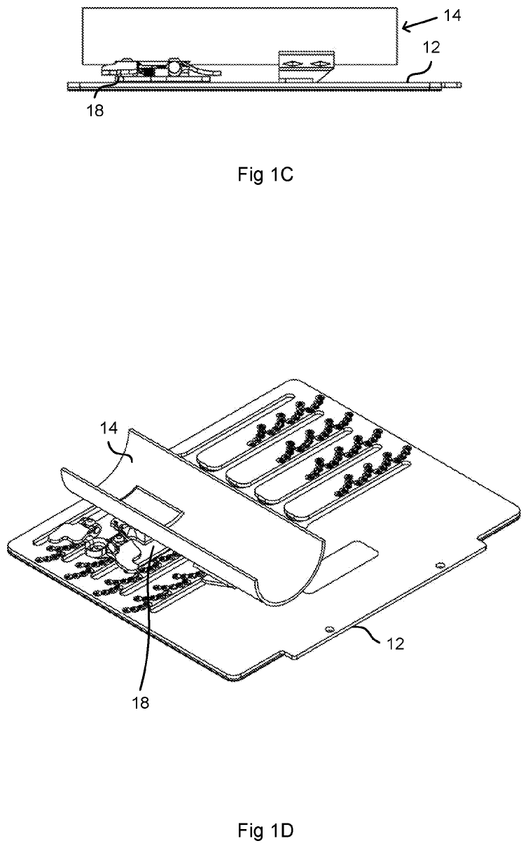

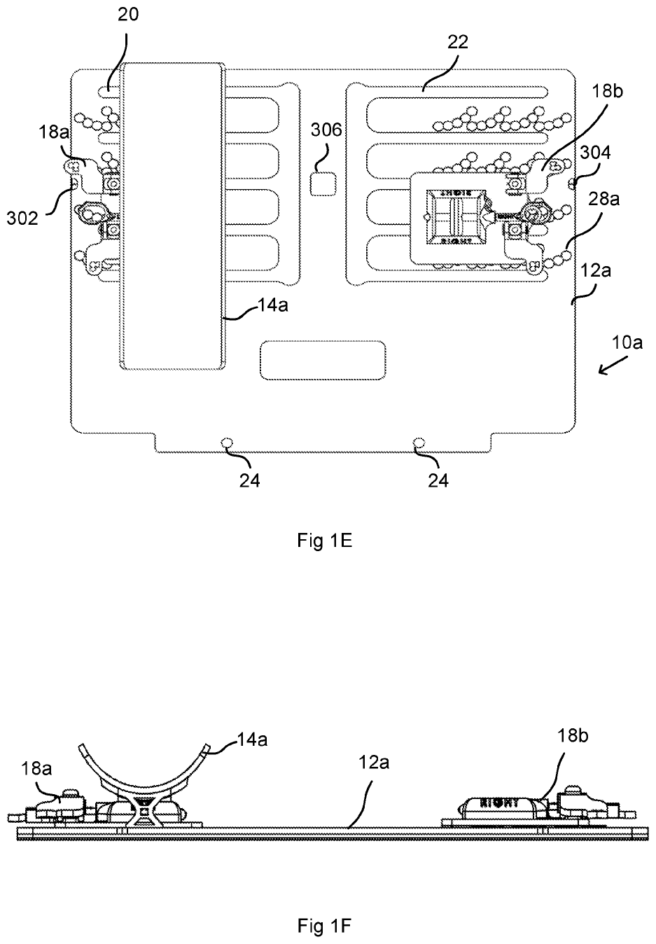

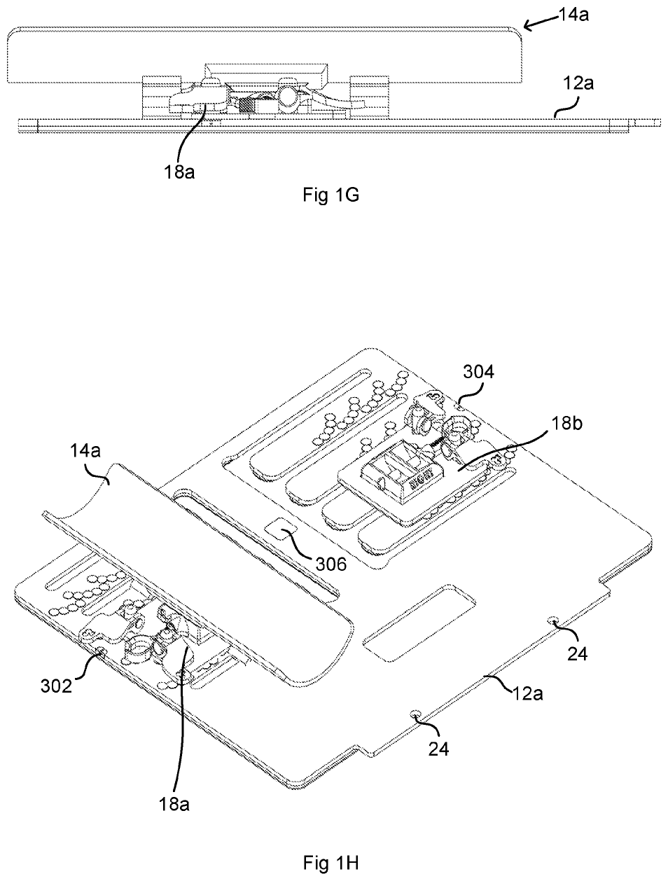

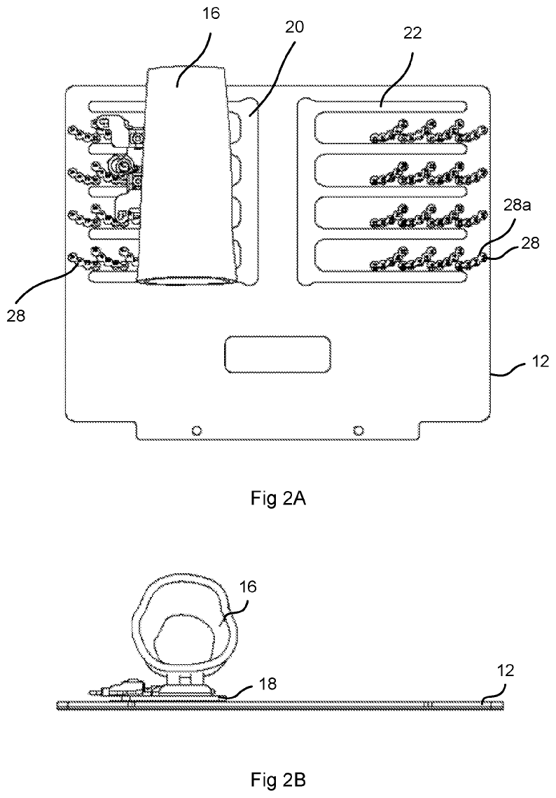

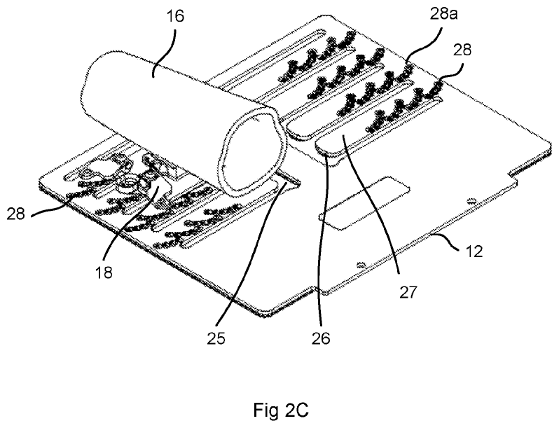

Referring first to FIGS. 1A to 1D and 2A to 2C, a bolus positioning system 10 includes an indexing or positioning plate 12, a C-channel or cradle component 14, a bolus component 16 and at least one locking mechanism. The at least one locking mechanism may be in the form of an interconnecting locking plate 18. The C-channel component 14 operates interchangeably with the bolus component 16 to accurately locate, support, immobilise and regulate radiation exposure to, for example, a patient's limb, when undergoing radiotherapy, usually in combination with a vacuum bag. The indexing plate 12 is formed with a plurality of multiposition-enabling formations and corresponding reference locations. The plurality of multiposition-enabling formations may be in the form of a series of locating tracks or channels 20 and 22 within which the locking plate 18 may locate and travel to allow adjustment of the bolus component 16 and C-channel component 14 in the x-y plane defined by the upper surface of the indexing plate 12. It will be appreciated that the multiposition-enabling formations may take many different forms of tracks, channels or guides which cooperate with the at least one locking mechanism to allow multipositional adjustment of the mechanism in the x-y plane.

The indexing plate 12 is in turn mounted transversely via locking bar locating holes 24 on a retaining mechanism (e.g. CIVCO Lok-Bar™, not shown) across a fixture, for example, a radiography treatment bed/couch (not shown). The indexing plate is manufactured from three layers of computer numerical control (CNC) milled radio-translucent carbon fibre sheet or other relatively rigid radio-translucent materials such as a Kevlar sheet or fibreglass sheeting. The locking plate 18 may be formed from a similar material, including carbon fibre sheeting, Kevlar sheeting or fibreglass sheeting. Various interconnects and levers featured on the locking plates may be manufactured from durable thermoplastics using traditional or advanced manufacturing techniques. The layers are laminated together to form a rigid structure. It will be appreciated that the number of layers may vary. The channels or slots 20 and 22 are milled through the upper two layers (intermediate layer 26 and upper layer 27), with a base layer 25 defining the channel floor. The intermediate layer 26 is recessed relative to the upper layer 27 in the region of the channels so as to define locating recesses. Also milled into the upper layer are the plurality of reference locations. In one example, the plurality of reference locations are a series of indexing indicia or co-ordinates 28 located in arcuate arrays of circular recesses 28a for providing reference positions for the bolus component 16 and C-channel or cradle component 14 for each user being treated. An alphanumeric coordinate numbering system is used (A1, B2, C3 etc), though any other suitable numbering or indexing system may be employed using suitable indicia.

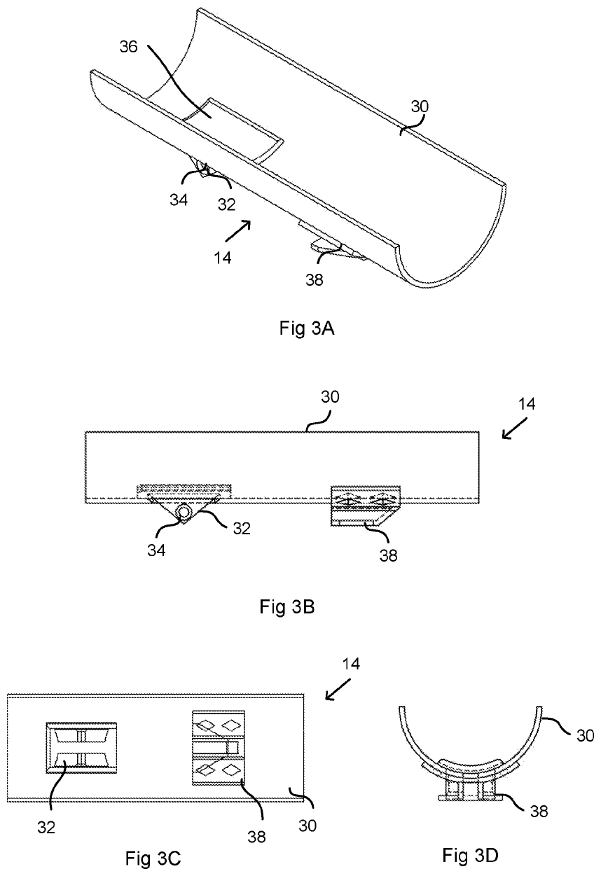

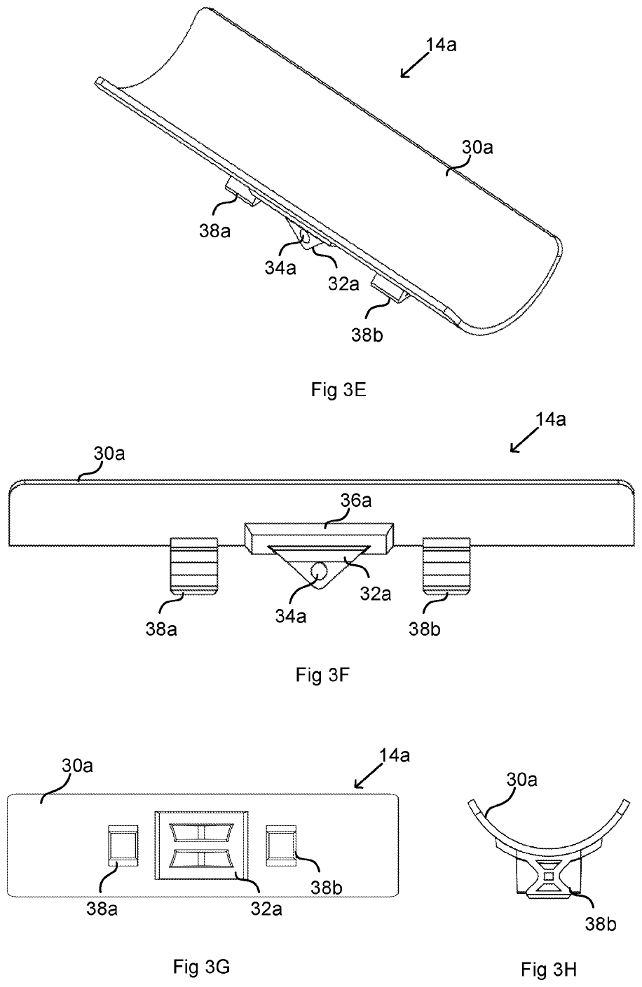

Referring now to FIGS. 3A-3D, the C-channel or cradle component 14 comprises a semi-cylindrical C-channel or cradle portion 30 formed from a transparent or translucent material. Perspex, polycarbonate and translucent polyvinyl chloride polymers which provide the desired radio translucent properties may be used.

The C-channel portion 30 locates and supports a custom sized vacuum bag (not shown). A limb is placed within the vacuum bag cavity from which air is drawn, immobilising the limb for the acquisition of user specific data using a 3D scanning method (e.g. computerised tomography (CT)) once the C-channel portion is mounted in position to the indexing plate in the manner shown in FIGS. 1A to 1D. The vacuum bag may immobilise the limb within the C-channel component in a position providing the user with comfort during later treatment, during which the position of the limb is immobilised within the personalised bolus (instead of the vacuum bag) that is created based on the user specific data obtained by the 3D scanning method. For example, limited tilting of the user's limb may be facilitated relative to the C-channel component. In this regard, the user limb may be supported at the most appropriate angle for the user and limb being treated. This angle may be replicated for creating the bolus and allows a variable bolus locking angle that is specific to the personalised bolus for the user. In addition, the vacuum bag separates the structure of the C-channel and that of the user limb, which facilitates accurate measurements of the user limb by the CT scan, as the vacuum bag provides an area of lower density which is more clearly visible within the CT scan.

Alternatively, the user limb may be placed within and supported by the C-channel without a vacuum bag during the CT scan. However, the colliding structures of the user limb and the C-channel may be indiscernible from the CT scan when they are in contact due to similarities in density. Manual manipulation of the user specific data may be required to redesign the portions affected. Alternatively, the C-channel may be formed from other appropriate radio translucent or transmissive materials having differences in density compared to the user limb in the CT scan to solve the indiscernibility problem as discussed above. The appropriate materials are required to be safe within a CT field for both the user and equipment.

Fixed to the base of the C-channel component 30 is a C-channel or cradle connector 32 having a V-profile with through-hole 34 for detachably and rigidly coupling to the locking plate 18. The cradle connector 32 is firmly anchored in position to C-channel portion via a backing plate 36. A support sled 38 is also fixed to the base of the C-shaped portion and provides additional load bearing support for the C-channel component 14 by resting on the upper surface of the indexing plate 12, as is shown in FIGS. 1A to 1D, so that the C-channel is parallel to the indexing plate. Both the cradle connector 32 and support sled 38 are formed by an additive manufacturing process such as 3D printing. It will be appreciated that they may also be manufactured using an extrusion method, injection moulding process, subtractive manufacturing including milling and carving, or any other suitable manufacturing process. It will also be appreciated that the manufacturing process may be selected from two or more processes described above.

It will further be appreciated that the cradle component 30 may be shaped to comfortably accommodate the opposite side of an affected area of any body part requiring treatment such as the ankle, hand, wrist or foot.

FIGS. 1E to 1H illustrate a second embodiment of a bolus positioning system 10a including an indexing or positioning plate 12a, a C-channel or cradle component 14a, and the at least one locking mechanism. As discussed above, the at least one locking mechanism may be in the form of the interconnecting locking plate (i.e. 18a or 18b shown in FIGS. 1E to 1H, which has the same structure and features as the interconnecting locking plate 18). In FIGS. 1E to 1H components and features of the bolus positioning system 10a similar to the bolus positioning system 10 described with reference to FIGS. 1A to 1D are shown with like reference numerals.

The bolus positioning system 10a is different from the bolus positioning system 10 described in FIGS. 1A to 1D in that the indexing plate 12a additionally includes at least a first set of cross hairs 302 and a second set of cross hairs 304 positioned at opposite edge of the positioning plate, respectively, and another aperture 306 positioned between the first set of cross hairs 302 and the second set of cross hairs 304. The cross hairs 302 and 304 may be printed on the indexing plate 12a. It will be appreciated that they may also be manufactured using an extrusion method, injection moulding process, subtractive manufacturing including milling and carving, or any other suitable manufacturing process. It will also be appreciated that the manufacturing process may be selected from two or more of the processes described above. In one example, the cross hairs 302, 304 and aperture 306 are in transverse alignment along a transverse line which is parallel to the upper edge of the indexing plate and may facilitate accurate and consistent positioning of the indexing plate 12a on the retaining mechanism (not shown) across the fixture (e.g. a radiography treatment bed (not shown)) by taking reference to laser beams directed through said apertures from a stationary overhead gantry. In other examples, other arrangements of the aligning cross hairs and apertures may be used to facilitate accurate and consistent positioning of the indexing plate 12a on the retaining mechanism across the fixture by taking reference to laser beams directed through said cross hairs and apertures. The cross hairs 302 and 304 alone (i.e. without the need of aperture 306) may also serve to provide a guide for the laser beams. During later treatment, the cross hairs and/or aperture(s) may also facilitate positioning a user's limb with the bolus and the indexing plate without the need for user tattoo markers for positioning.

The bolus positioning system 10a is also different from the bolus positioning system 10 described in FIGS. 1A to 1D in relation to a second embodiment of a C-channel or cradle component 14a. Referring now to FIGS. 3E-3H, the C-channel or cradle component 14a comprises a semi-cylindrical C-channel or cradle portion 30a formed from a transparent or translucent material. Fixed to the base of the C-channel portion 30a is a C-channel or cradle connector 32a having a V-profile with a through-hole 34a for detachably and rigidly coupling to the locking plate 18a. The C-channel connector 32a is firmly anchored in position to C-channel portion 30a via a backing plate 36a positioned substantially centrally on the base of the C-channel portion 30a. The substantially central position of the C-channel connector 32 may facilitate placement of the locking plate 18a more centrally under the limb for CT scanning. The backing plate 36a positioned on the base of the C-channel portion 30a and not projecting into the inner limb receiving surface of the C-channel portion may facilitate eliminating the possibility of the non-projecting backing plate intersecting the limb (or any body part requiring treatment) in the CT scanning output.

At least two support sleds 38a and 38b are also fixed to the base of the C-channel portion 30a and provide additional load bearing support for the C-channel component 14a by resting on the upper surface of the indexing plate 12a, as is shown in FIGS. 1E to 1H, so that the C-channel component 14a is parallel to the indexing plate 12a. Both the cradle connector 32a, backing plate 36a and support sleds (38a and 38b) are formed by an additive manufacturing process such as 3D printing. It will be appreciated that they may also be manufactured using an extrusion method, injection moulding process, subtractive manufacturing including milling and carving, or any other suitable manufacturing process. It will also be appreciated that the manufacturing process may be selected from two or more of the processes described above.

It will further be appreciated that the cradle component 30a may be shaped to comfortably accommodate the opposite side of an affected area of any body part requiring treatment such as the ankle, hand, wrist or foot.

Although FIGS. 1A-1D illustrate the bolus positioning system 10 including the C-channel component 14 and the indexing plate 12 while FIGS. 1E to 1H illustrate the bolus positioning system 10a including the C-channel component 14a and the indexing plate 12a, it is appreciated that the C-channel component 14a may co-operate with the positioning plate 12 and that the C-channel 14 may co-operate with the positioning plate 12a.

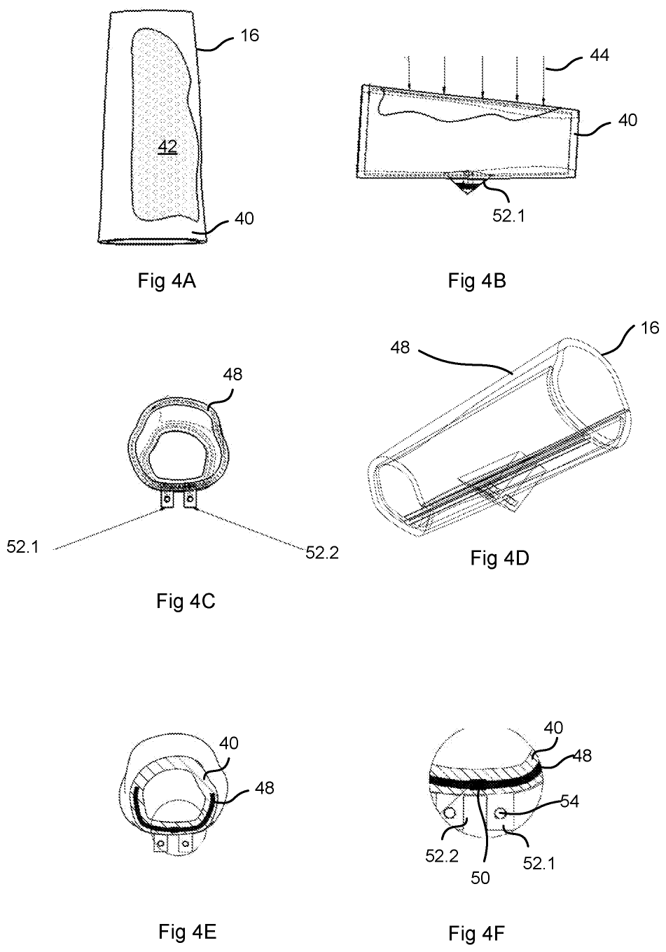

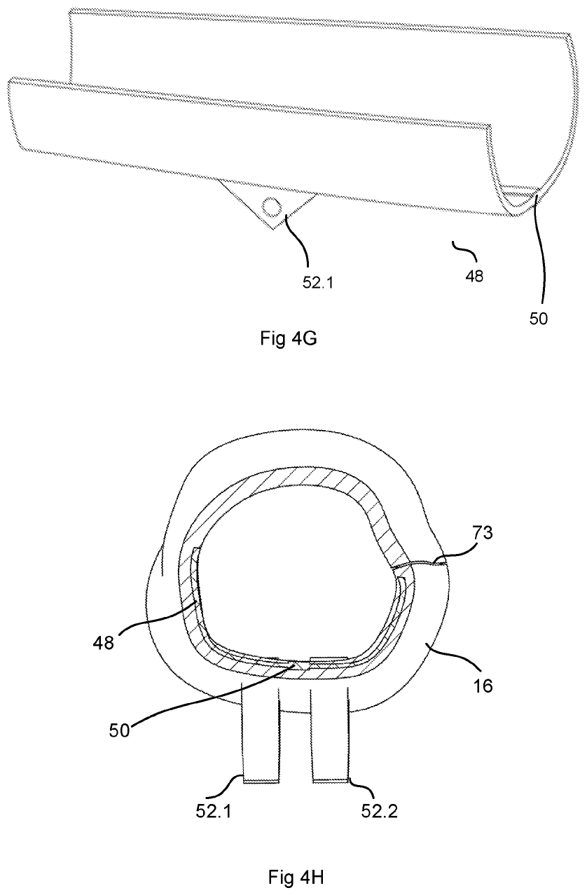

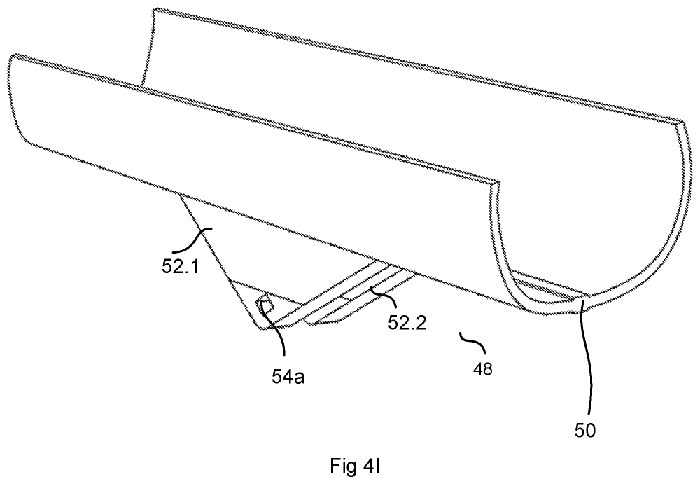

Referring now to FIGS. 4A to 4F, the bolus 16 is shaped and customised to snugly fit the limb of a particular user requiring treatment, such as their forearm. The bolus 16 includes a relatively non-rigid skin interfacing layer. The skin interfacing layer may be formed by a soft overmoulded skin-interfacing material 40 such as silicone, having a shore A hardness between 10 and 60 using a Shore durometer. It will be appreciated that numerous other suitable relatively soft tissue-equivalent materials with similar properties may be used such as polyurethane, thermoplastic-waxes, and latex rubbers. In this embodiment, the bolus 16 has a uniform thickness. A radiation treatment area 42 is targeted via a radiation treatment planning system. The radiation treatment area 42 has similar properties to the rest of the bolus, including thickness, with the radiation beams being typically normal to the surface being treated, as is shown at 44.

As is clear from FIGS. 4D to 4F, the overmoulded material 40 is moulded over a relatively rigid endoskeleton structure 48 which may be 3D printed from a relatively hard polymer such as Polylactic Acid (PLA), Acrylonitrile Butadiene Styrene (ABS), or another suitable thermoplastic. In one example, the endoskeleton structure 48 may be extended substantially around the user limb. FIGS. 4B and 4C illustrate an example where the endoskeleton structure 48 is extended all the way around the user limb. In another example, the endoskeleton structure 48 may be extended so as to substantially wrap around the base area of the bolus, as shown in FIG. 4H. It will be appreciated that the endoskeleton structure 48 may also be manufactured using an extrusion method, an injection moulding process, subtractive manufacturing including milling and carving, or any other suitable manufacturing process. It will also be appreciated that the manufacturing process for the endoskeleton structure 48 may be selected from two or more processes described above. For example, the endoskeleton structure 48 may first be 3D printed or moulded and then milled to achieve dimensional accuracy.

The endoskeleton structure 48 may be formed with one or more reinforcing ribs 50, and may generally take the form of a solid or consistent/uniform structure. The endoskeleton structure 48 may produce an internal support of the desired thickness at the bolus midpoint. In one example, the internal support is provided on the underside of the limb, typically extending around the operatively lower half of the bolus to facilitate placement of user's limb, as is shown in FIGS. 4E and 4F. This thickness of the endoskeleton should provide adequate rigidity but not adversely interfere with the softness and pliability of the relatively non-rigid skin interfacing layer. The supporting function of the endoskeleton means that the relatively non-rigid skin interfacing layer may be formed from a softer and more pliable material, providing additional comfort to the user wearing the bolus 16, without compromising support.

At least one interconnecting formation is provided on the bolus 16 for rigidly interconnecting the at least one locking mechanism (e.g. the locking plate 18) and the bolus 16 in a recordable position for enabling repeated radiotherapy treatments on the same target body portion of the user in the same position. In one example, the at least one interconnecting formation includes at least one bolus connector extending from the endoskeleton structure 48. As shown in FIG. 4C, extending from and formed integrally with the endoskeleton 48 are a pair of bolus interconnectors 52.1 and 52.2 having a V-profile and formed with through-holes 54 to enable the bolus to be detachably and rigidly coupled to the locking plate 18 in a manner to be described. The interconnectors are formed at exactly the same location as the corresponding interconnectors 32 of the C-channel component 14.

FIG. 4G shows a more detailed perspective view of the endoskeleton structure 48 which has a “halfpipe” configuration and is formed with the central reinforcing rib 50 and the interconnectors, one of which is shown as 52.1.

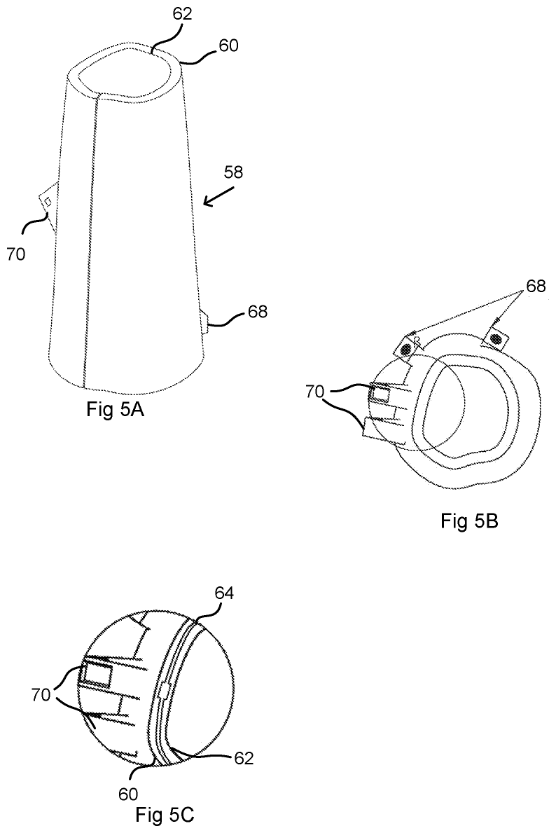

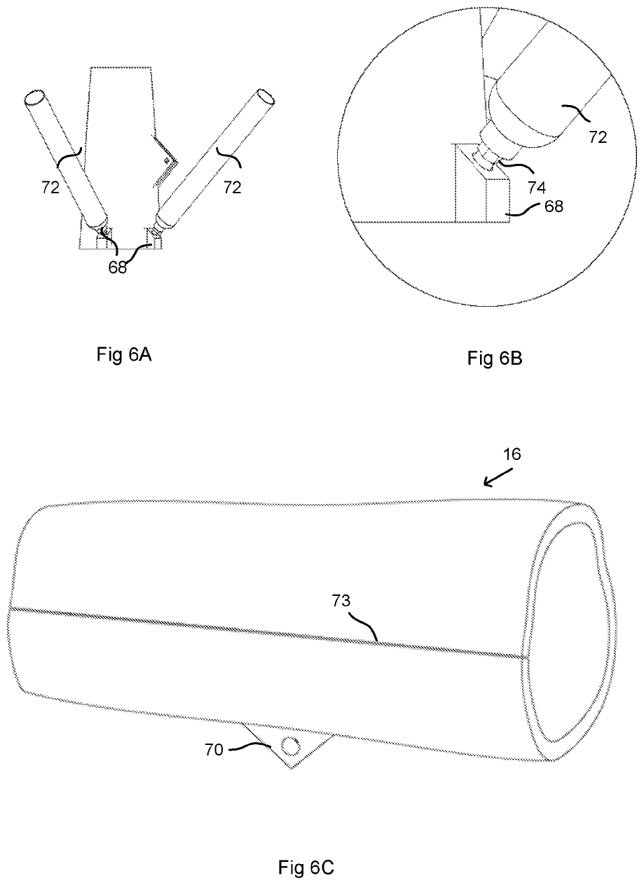

Referring now to FIGS. 5A to 5C, a further embodiment of a bolus component 58 shown in the process of manufacture. During this process, the bolus component 58 is encapsulated by a moulding shell comprising outer and inner walls 60 and 62 typically formed from the same or different material by and defining a moulding cavity within which is located a ribbed reinforcing endoskeleton 64. At least one injection port 68 is formed at the outer shell 60 and a pair of bolus interconnectors 70 extend from the endoskeleton 64 midway along the outer shell 60. For example, a pair of injection ports 68 is formed close to one end of the moulding shell as illustrated in FIGS. 5A, 6A and 6B. The outer and inner shells 60 and 62, the bolus connectors 70, the injection ports 68 and the ribbed reinforcing endoskeleton 64 may all be formed integrally by an additive manufacturing process such as 3D printing in a manner that will be described further on in the specification.

In another example, the outer and inner shells 60 and 62, the bolus connectors 70, the injection ports 68 and the ribbed reinforcing endoskeleton 64 may be formed separately from different materials and bonded in place. It will be appreciated that they may also be manufactured using an extrusion method, injection moulding process, subtractive manufacturing including milling and carving, or any other suitable manufacturing process. It will also be appreciated that the manufacturing process may be selected from two or more processes described above.

FIG. 6A shows how syringes 72 are fitted into the injection ports 68 via suitable adapters 74 shown in detail in FIG. 6B. The syringes 72 are filled with silicone which is injected into the moulding cavity to fill it entirely to form the overmoulded silicon layer. Once the silicone has cured, the outer and inner polymer walls 60 and 62 of the shell are cut away, together with the injection ports 68.

As shown in FIG. 6C (or FIG. 4H), the relatively non-rigid resilient skin interfacing layer of the bolus 16 may be provided with a longitudinally extending cut 73 which is shown to be perpendicular but may also be angled. This provides a clamshell-type opening to facilitate the bolus to be opened and snugly and accurately fitted in place around the limb of the user. This ensures consistent coverage of the skin and also serves as a reference for the degree of swelling.

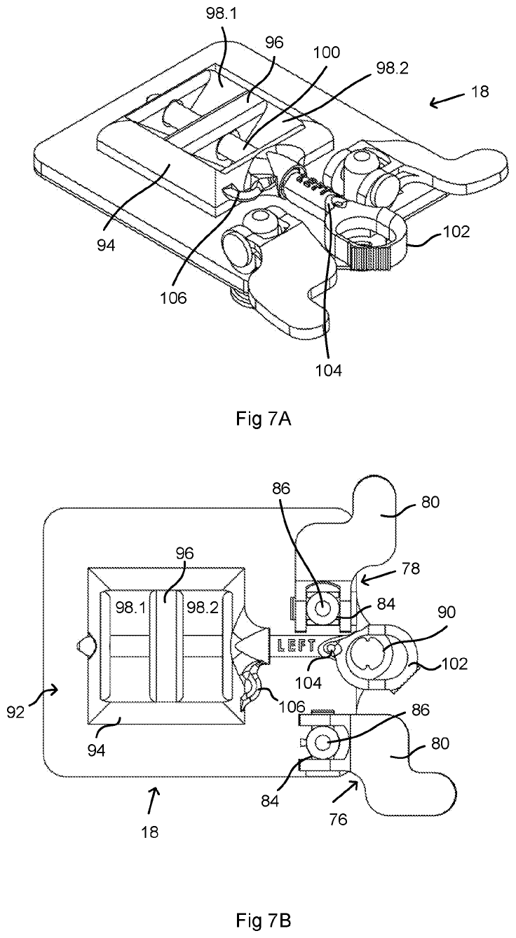

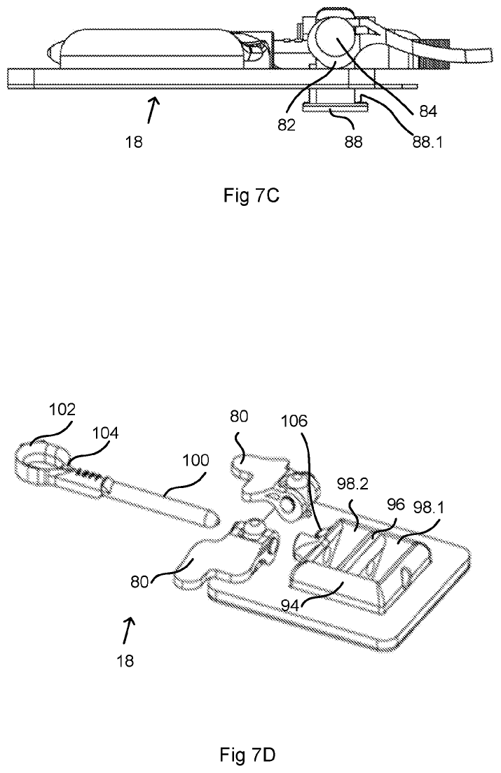

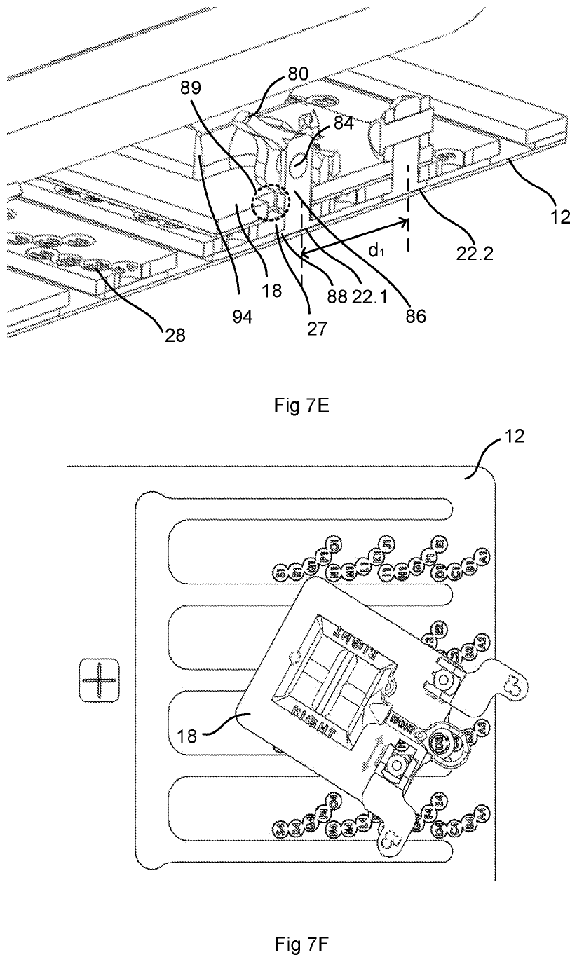

FIGS. 7A to 7F are detailed views of the locking plate 18, which includes a pair of clamping assemblies 76 and 78, each of which are provided with a locking lever 80 having a snail cammed or eccentric portion 82 which pivots around shaft 84. The shaft 84 in turn extends through stem 86 which terminates in a locking foot 88. Pivoting of the locking lever 80 between the open and closed positions causes the foot 88 to be urged upwards by virtue of the interaction of the cammed portion 82 with the upper surface of the locking plate. With the lever 80 in the downward closed position, the cammed portion 82 is in the over-centre position so as to ensure the lever is firmly biased closed.

The upper surface 88.1 of the locking foot 88 bears up against an underlying surface of the upper layer 27 of the indexing plate 18 so as to clamp the locking plate 18 in position. The centre-to-centre distance dl between the respective stems 86 of the locking levers 80 is essentially the same as the centre-to-centre distance between adjacent channels 22.1 and 22.2. The locking plate 18 may further include a slot 89 around the stem 86 as illustrated in FIG. 7E, which allows both linear and angular movement of the locking plate 18 for greater freedom of movement to the desired position prior to locking. FIG. 7F illustrates one example of the angular movement configuration of the locking plate 18 controlled by the slot 89 on the locking plate. The movement of the locking plate 18 may be limited to up to 45 degrees of movement in each direction.

It will be appreciated that various other types of locking arrangements may be used to lock, unlock and adjust the locking plates 18 relative to the channels 20 and 22 in the indexing plate 12, including those based on clamping screws for example.

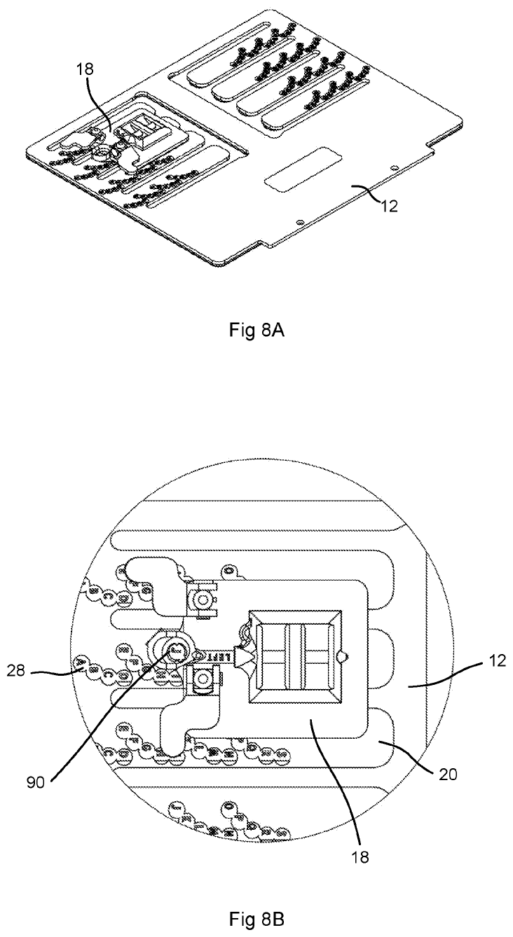

Extending between the locking levers 80 is an index coordinate viewing window 90 through which the indexing coordinates 28 may be viewed or sighted and aligned in moving the locking plates into the desired position and then recording that position based on the sighted indexing coordinate. FIGS. 8A to 8C show the locking plate 18 locked firmly within the indexing plate 12, with indexing co-ordinate J viewable through the viewing window 90.

The locking plate is further provided with a complemental connecting assembly 92 for receiving the bolus and C-channel connectors. The connecting assembly 92 includes at least one complemental connector for interlocking with the corresponding V-shaped bolus connectors 70 and C-channel connectors 32. In one example, the complemental connector is in a form of a surround 94 with a central divider 96 defining a pair of V-slots the 98.1 and 98.2 as shown in FIG. 7A within which the corresponding V-shaped bolus connectors 70 and C-channel connectors 32 locate. A locking formation may be provided for rigidly interlocking the bolus (or the C-channel components) and complemental connector. In one example, the locking formation is in a form of a locking pin 100. In particular, the locking pin 100 extends through apertures in the surround 94 and central divide 96 of the connecting assembly 92 as shown in FIGS. 7A and 7D. In order to lock the bolus component 16 or C-channel component 14 in position, the locking pin 100 is retracted via an integral ring pull 102 to a position indicated in FIG. 7D. The V-shaped bolus or C-channel connectors 70 and 32 are nested within the complemental V-slots 98.1 and 98.2, and the pin 100 is inserted through the aligned apertures in the V-slots and corresponding V-shaped connectors to lock the bolus or C-channel component firmly in position as per FIGS. 1 and 2. The locking pin 100 is provided with a tethering loop 104 and corresponding tethering loop 106 is provided on the surround 84 to prevent the locking pin 100 being misplaced via a tether (not shown). In addition, the viewing window 90 as shown in FIG. 8B may be configured as a through-hole. Another locking or aligning pin (not shown) may be inserted through the viewing window 90 to further align the locking plate 18 (or 18a or 18b) with a selected corresponding indexed recess 28a in the indexing plate 12 (or 12a).

It will be appreciated that the V-slots and corresponding connectors may have other complemental configurations which allows them to be locked firmly in position onto the locking plate as well as being unlocked with ease. These may in particular include complemental profiles which are able to engage and disengage by upward and downward movement substantially normal to the plane of the locking and indexing plate. This corresponds to natural raising and lowering movement of the limb, which includes pre-alignment, as opposed to an arrangement requiring lateral location and movement such as a dovetail-type connection which is generally less conducive to natural movement and pre-alignment. A self-centering arrangement such as that provided by the V-slots and corresponding connectors is also suited to making allowance for imprecise pre-alignment.

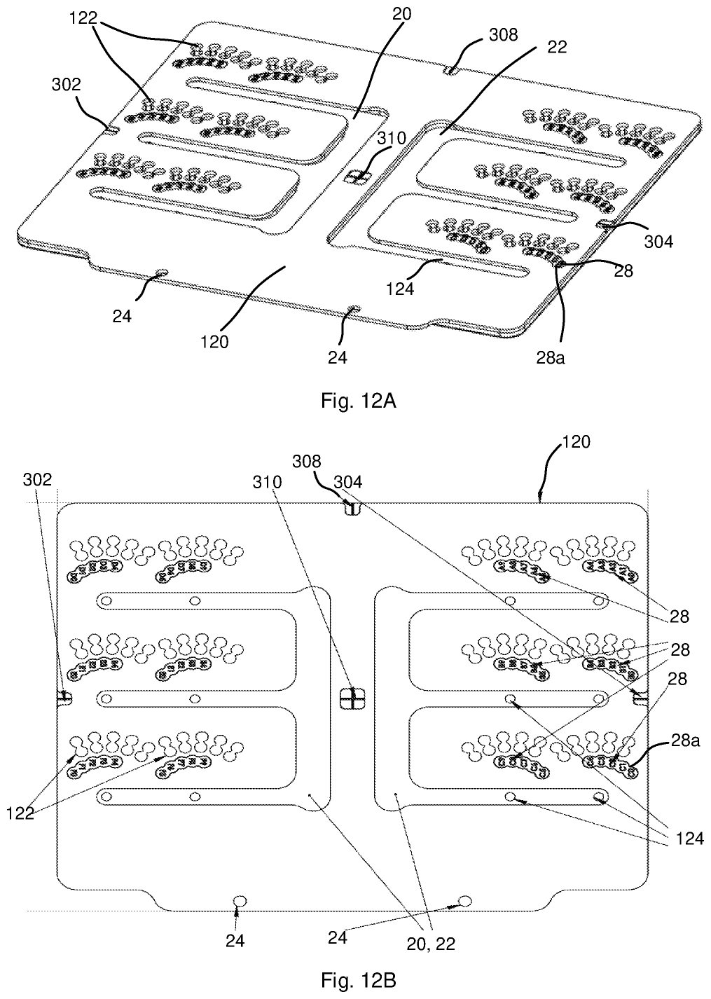

FIGS. 12A and 12B illustrate another embodiment of an indexing plate 120. Components and features of the indexing plate 120 similar to the indexing plates 12 and 12a described with reference to FIGS. 1A to 1H and FIGS. 2A to 2C are shown with like reference numerals.

In addition to the locating tracks or channels 20 and 22, the locking bar locating holes 24, the indexing indicia or co-ordinates 28, the recesses 28a and the cross hairs 302 and 304, the indexing plate 120 includes the plurality of multiposition-enabling formations in the form of a series of locating pin holes 122 having a figure-of-eight configuration. It will be appreciated that other hole profiles may be used in combination with complemental pin profiles. In some examples, the locking plate 120 may also include one or more feedback positioning holes 124 within one or more of the locating tracks or channels 20 and 22. The feedback positioning holes 124 may provide tactile feedback to the user when moving the locking foot 88 of the locking plate along the locating tracks or channels 20/22. The aperture 306 as shown in FIG. 1E may be omitted from the indexing plate 120. The indexing plate 120 may include additional markers (e.g. in the form of cross hairs 308 and 310 as illustrated in FIGS. 12A and 12B) for providing a guide for the laser beams. As mentioned previously, during laser treatment, the cross hairs may also facilitate positioning a user (e.g. patient)'s limb with the bolus and the indexing plate without the need for user tattoo markers for positioning.

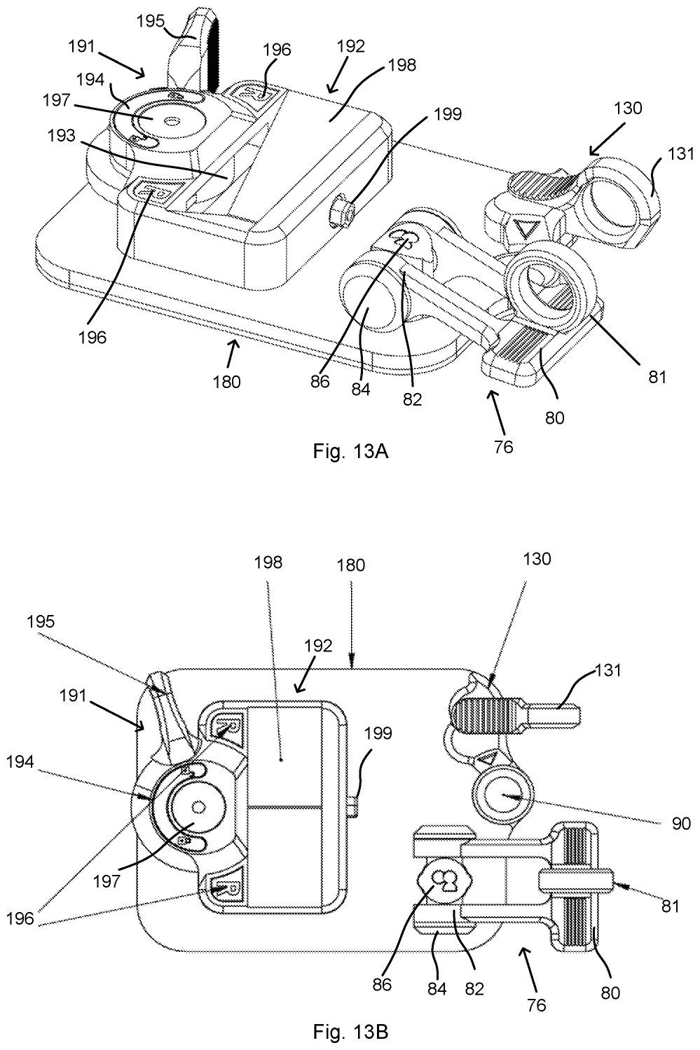

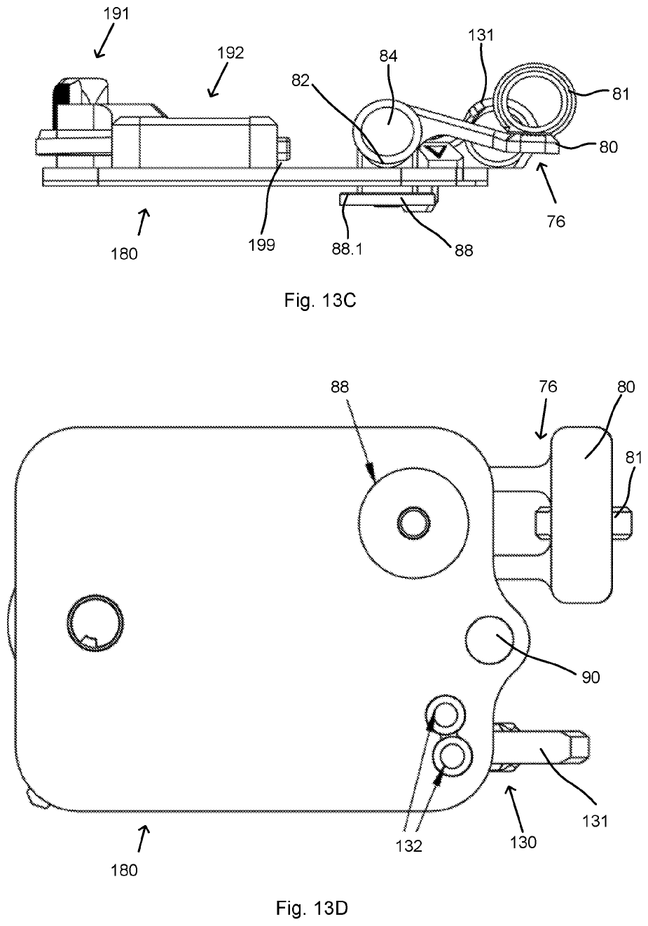

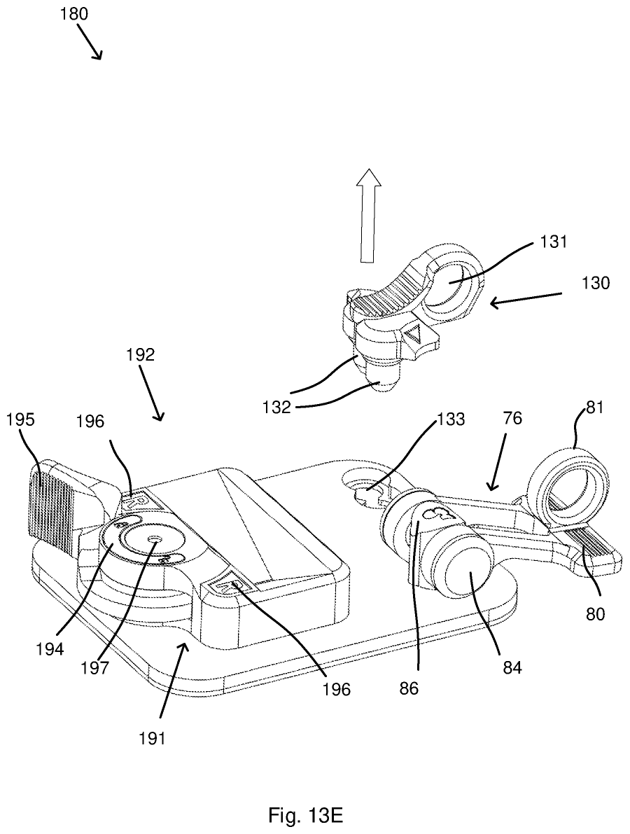

FIGS. 13A-13E illustrate detailed views of another exemplary locking plate 180 designed to cooperate with the indexing plate 120 of FIGS. 12A and 12B. In FIGS. 13A-13E components and features of the locking plate 180 similar to the locking plate 18 described with reference to FIGS. 7A to 7F and FIG. 8B are shown with like reference numerals. In particular, FIGS. 13A-13E illustrate respective perspective, top plan, front and underplan views of the locking plate 180.

The locking plate 180 includes a clamping assembly 76 for engaging with the locating tracks or channels 20 and 22 in the indexing plate 120. The clamping assembly 76 is provided with a locking lever 80 having a snail cammed or eccentric portion 82 which pivots around a shaft 84. The shaft 84 in turn extends through stem 86 which terminates in a locking foot 88. Pivoting of the locking lever 80 between the open and closed positions causes the foot 88 to be urged upwards by virtue of the interaction of the cammed portion 82 with the upper surface of the locking plate. With the lever 80 in the downward closed position, the cammed portion 82 is in the over-centre position so as to ensure the lever is firmly biased closed. The clamping assembly 76 may also include a handle 81 for facilitation of user engagement with the locking lever 80. Details of the engagement between the clamping assembly 76 with the locating tracks or channels 20 and 22 are described with reference to FIG. 7E.

The locking plate 180 may also include a locating pin assembly 130 for engaging with the locating pin holes 122 of the indexing plate 120. In particular, the locating pin assembly 130 includes at least one locating pin 132 for positioning the locking plate 180 at a desired locating pin hole 122 which corresponds to an indexing coordinate 28. The locating pin assembly 130 may be removed (i.e. unplugged, see FIG. 13E) from the locating late 180 until the desired position (including angular position) is selected. The locating pin assembly 130 is then pressed through a locking plate pin hole 133 and into the respective locating pin holes 122 of the indexing plate 120 for locating the locating plate 180 at the desired position. The locating plate 180 can then be secured through the clamping assembly 76. The locating pin assembly 130 may also include a ringpull handle 131 for facilitation of finger and thumb engagement with the locating pin assembly 130.

Extending between the clamping assembly 76 and the locating pin assembly 130 is an index coordinate viewing window 90 through which the indexing coordinates 28 may be viewed or sighted and aligned in moving the locking plates into the desired position and then recording that position based on the sighted indexing coordinate. In some embodiments each indexing coordinate corresponds to a locating pin hole 133. In some embodiments, the viewing window 90 may be part of the locating pin assembly 130.

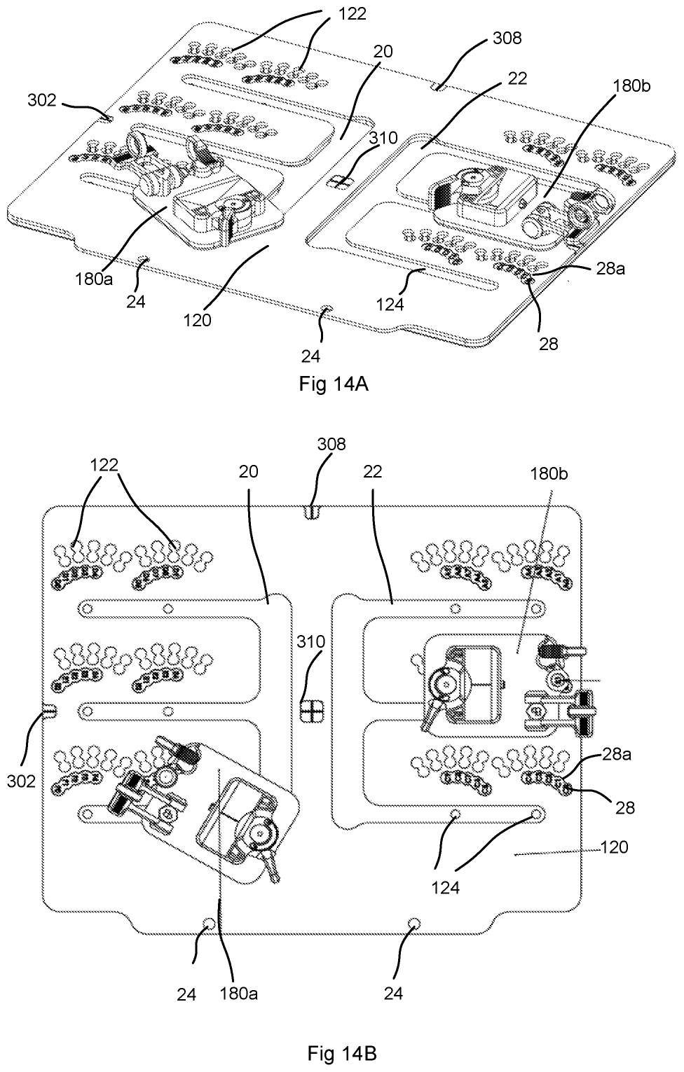

The locking plate 180 allows both linear and angular movement for greater freedom of movement to the desired position prior to locking. FIGS. 14A and 14B illustrate respective perspective and top plan views of an exemplary angular configuration of a locking plate 180a and an exemplary non-angular configuration of a locking plate 180b with the indexing plate 120. The movement of the locking plate 180 may be limited to 5 angular positions, 15 degree increments per indexing location for a total of up to 60 degrees of movement. This can clearly be seen in FIGS. 14A and 14B where the 60 degree arcs of five locating pin holes 122 and corresponding indexing coordinates and recesses 28 and 28a allow for 60 degrees of movement at 15 degree intervals. In this regard, it will be appreciated other angular positions and/or degrees of angular movement can be allowed.

It will be appreciated that various other types of locking arrangements may be used to lock, unlock and adjust the locking plates 180 relative to the channels 20 and 22 in the indexing plate 120, including those based on clamping screws for example. In one example, the disclosed locking plate 180 and the indexing plate 120 may provide 60 distinct lockable positions for various attachable accessories including the disclosed C-channel components and bolus components.

The locking plate 180 is further provided with a complemental connecting assembly 192 for receiving the bolus and/or C-channel connectors. The connecting assembly 192 includes at least one complemental connector for interlocking with the corresponding V-shaped bolus connectors and C-channel connectors. In this example, the complemental connecting assembly 192 is in a form of a V-slot 198 as shown in FIGS. 13A and 13B within which the corresponding V-shaped bolus connectors and C-channel connectors locate.

A locking formation may be provided for rigidly interlocking the bolus (or the C-channel components) and complemental connector. In this example, the locking formation is in a form of a rotating lock assembly 191. In particular, the received connector (e.g. of the C-channel component or the bolus) is locked by rotating a cam lock 193 of the rotating lock assembly 191 around a shaft 197 in a locked position. To unlock the received connector, the cam lock 193 is rotated in an opposite direction to the locked position. It will be appreciated that the locking formation, V-slot and corresponding connectors to be received by the locking formation may have different complemental configurations which allows them to be locked firmly in position onto the locking plate as well as being unlocked with ease.

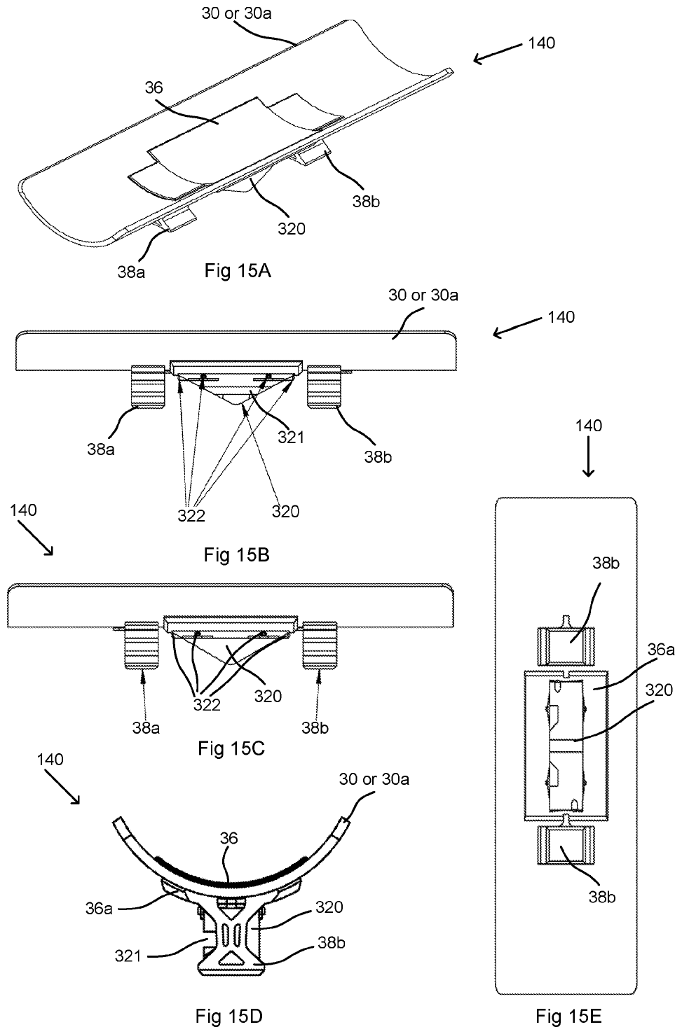

FIGS. 15A-15E illustrate respective perspective, first side, second side, end-on and underplan views of another exemplary C-channel component 140 for, for example, interconnecting with the locking plate 180. In FIGS. 15A-15E components and features of the C-channel component 140 similar to the C-channel component 14a described with reference to FIGS. 3E to 3H are shown with like reference numerals. The C-channel component 140 differs from the C-channel component 14a primarily in the connector for engaging with the locking plate. In particular, the C-channel component 140 includes a C-channel connector 320 (e.g. in V-profile) fixed to the base of the C-channel portion 30 or 30a for detachably and rigidly coupling to the locking plate 180. The C-channel connector 320 may include a holding slot 321 for receiving the cam lock 193 of the rotating lock assembly 191 in the locked state. The C-channel connector 320 may also include one or more CT-distinguishable markers 322 (e.g. ceramic markers) for aid in aligning the bolus during data processing stage. In some examples, the one or more CT-distinguishable markers may be manufactured from a material denser than surrounding material, such as ceramic alumina oxide, which provide more easily discernible indication of position of the one or more CT-distinguishable markers and hence the C-channel component 140 when CT data is interpreted.

The substantially central position of the C-channel connector 320 may facilitate placement of the locking plate 180 more centrally under e.g. the limb for CT scanning. Both the C-channel connector 320, backing plate 36a and support sleds (38a and 38b) can be formed by an additive manufacturing process such as 3D printing. It will be appreciated that they may also be manufactured using an extrusion method, injection moulding process, subtractive manufacturing including milling and carving, or any other suitable manufacturing process. It will also be appreciated that the manufacturing process may be selected from two or more of the processes described above.

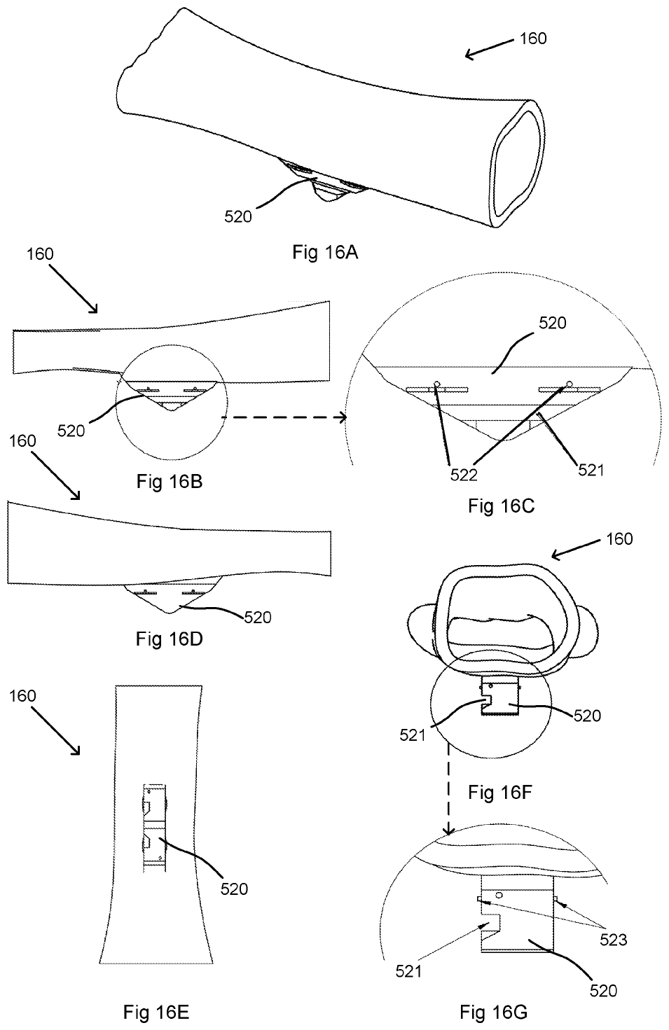

FIGS. 16A-16G illustrate respective perspective, first side, detailed first side, second side, underplan, end-on and detailed end-on views of another exemplary bolus component 160. In FIGS. 16A-16G components and features of the bolus component 160 similar to the bolus component 16 described with reference to FIGS. 4A to 4F are shown with like reference numerals. The bolus component 160 differs from the bolus component 16 primarily in the connector for engaging with the locking plate. In particular, the bolus component 160 includes a bolus connector 520 (e.g. in V-profile) for detachably and rigidly coupling to the locking plate 180. The bolus connector 520 may be extended from and formed integrally with the endoskeleton (not shown). The bolus connector 520 is formed at exactly the same location as the corresponding C-channel connector. The bolus connector 520 may include a holding slot 521 for receiving the cam lock 193 of the rotating lock assembly 191 in the locked state. The bolus connector 520 may also include one or more levelling tabs 523 that in combination provide an aligned surface for part of the clamping force to be applied and aid in consistent alignment of the bolus connector 520 and bolus component 160 in the complemental connecting assembly 192 of the locking plate 180. In some embodiments, the bolus component 160 may also include two blank holes 522 (as illustrated in FIG. 16C) for alignment purposes.

Referring back to FIGS. 13A-13E, the rotating lock assembly 191 may also include an indicator 194 indicating the lock/unlock state of the cam lock 193. The rotating lock assembly 191 may also include a lock lever 195 to facilitate the rotation of the cam lock 193. It will be appreciated that the locking plate can be manufactured in left and right configurations (e.g. to be used on the left/right side of the indexing plate e.g. for a left/right limb). In some embodiments, the locking plate 180 may also include at least one left/right side indicators 196. In the example shown in FIGS. 13A, 13B and 13E, the rotating lock assembly 191 includes two indicators 196 indicating the right side configuration.

In some embodiments, the locking locating pin assembly 130 may be provided with a tethering means (not shown) and corresponding tethering means 199 (e.g. in a form of a tethering plug) may be provided on the complemental connecting assembly 192 to prevent the locking locating pin assembly 130 being misplaced via e.g. a tether cord (not shown).

Although the disclosed indexing plates and locking plates are described for immobilisation of the bolus and/or C-channel, it will be appreciated that the disclosed locking plates may interface between the disclosed indexing plates and other accessories/devices that require immobilisation, accurate and/or repeatable positioning. It will also be understood that the indexing plates, locking plates, bolus components, C-channel components disclosed and defined in this specification extends to all alternative combinations of two or more of the individual features mentioned or evident from the text or drawings. In one example, the locking plate 180 may exclude the complemental connecting assembly 192 and the rotating lock assembly 191 but include the complemental connecting assembly 92 and the locking pin 100 for receiving and locking the C-channel connector 32 and/or bolus connectors (70, 52.1 and 52.2). All of the different combinations constitute various alternative aspects of the invention.

The method of manufacturing the bolus and other components of the system will now be described in more detail.

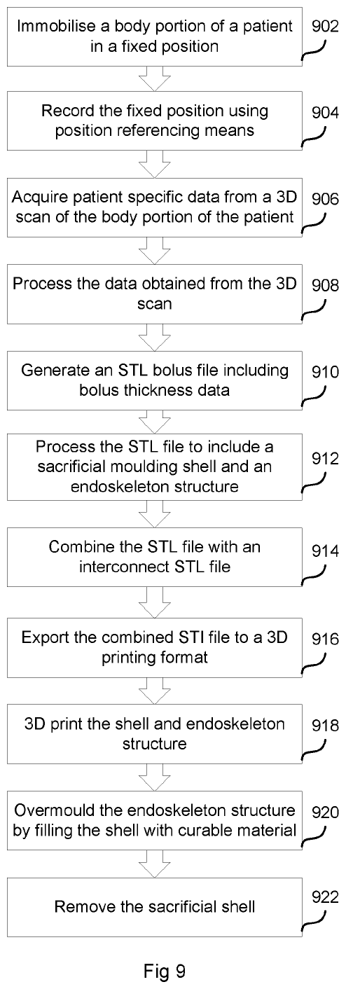

As broadly illustrated in FIG. 9, a body portion of a user (e.g. patient) is immobilised in a fixed position at step 902. The fixed position is then recorded using position referencing means at step 904. At step 906, patient specific data is acquired from a 3D scan of the body portion of the patient in the recorded fixed position. More specifically, in the case of the affected area being on a patient's limb, such as their forearm, patient specific limb data is acquired from a CT scan (or other 3D scan method) of the limb. This data is captured after placing a vacuum bag around the patient's forearm, placing the patient's forearm in the C-channel which has been attached to the indexing plate in roughly the correct position, immobilising the patient's forearm within the vacuum bag in the C-channel, fine adjusting the position of the C-channel on the indexing plate and locking it in place. This process also allows for the recording of the interconnect position relative to the limb by viewing the relevant co-ordinates through the viewing window in the locked position and recording these. This reference is critical for subsequently locating the bolus interconnect so that it positions the limb for radiation therapy in the same position as the CT scan.

At step 908, the patient specific data obtained from the 3D scan is processed. In one example, DICOM data obtained from the CT scan is processed in a DICOM-compatible file processing computer program to identify the skin boundary, the relative location of the C-channel interconnect and the desired radiation treatment area. The bolus thickness is determined by the treating physician in accordance with relevant standard and an offset from the skin boundary is set accordingly in this software. This allows for the automated generation of an STL bolus file at the desired thickness at step 910. The STL bolus file at the desired thickness may also include required markers including the C-channel location based on the co-ordinates and the radiation exposure zone.

At step 912, the STL bolus file is further digitally processed to create a moulding cavity representative of the desired bolus thickness. This is done by offsetting the skin surface face (inwards away from the patient's skin surface) and offsetting the outer bolus face outwards (away from the patient's skin). This creates a mould shell of the predetermined thickness. The offset amount should produce a thickness that is adequate for holding silicone and maintaining form during the moulding process but should still remain removable once 3D printed. This thickness should be thin enough to allow the mould shell to be easily removed but thick enough to maintain form and prevent moulding material from leaking out. Thickness may accordingly vary from around 0.7 mm to 1.5 mm. This range is selected to provide an appropriate balance between time to print, structural integrity of the mould shell for casting, and ease of mould shell to remove after the bolus material is cured. The mould shell may have a larger thickness of up to about 2 mm. It will be appreciated that the thicker the mould shell, the harder it will be to remove and the greater the risks of potentially damaging the bolus during the removal process.

At step 912, the STL bolus file is also processed to include an internal endoskeleton support structure. The internal endoskeleton support structure is designed by offsetting faces from the skin barrier to produce an internal supporting endoskeleton structure of the desired thickness at the bolus midpoint on the underside of the limb. This thickness should provide adequate rigidity but not adversely interfere with the softness and pliability of the silicone material.

At step 914, the resulting STL file, containing both the sacrificial moulding shell and the endoskeleton structure geometry is then virtually combined with the pre-designed standard interconnect STL file via the use of various Boolean/intersection operations which will be familiar to a person involved in CAD/3D design. The interconnect model is positioned as to align the limb in the same orientation and position as previously planned for during the CT scan.

At step 916, the combined STL file is ready to be exported to a slicing program for 3D printing. Parameters are set to minimise the need for support structures, increase print speed, optimise infill density and optimise surface finish.

At step 918, the printing process then commences and produces the desired bolus mould and integrated endoskeleton support structure. The bolus mould is post-processed, involving removing support and checking dimensional accuracy. The through-holes 54 formed with the bolus interconnectors 52.1 and 52.2 may be initially printed in a diamond or square shape 54a as illustrated in FIG. 41. The diamond or square shaped holes 54a may be then used as a drilling guide so that the through-holes 54 can be drilled with a drill bit with a certain diameter (e.g. a 10 mm drill bit). This process may facilitate maintaining consistency in diameter and positioning of the through-holes across the bolus.

At step 920, the endoskeleton structure is overmoulded by filling the shell with curable material. For example, the bolus mould is filled with silicone or another suitable curable tissue equivalent material via injection ports in the manner described above. In one example, the syringes 72 are manufactured independently with the injection ports 68 and the fitted into the injection ports 68 for injecting the overmoulded material into the moulding cavity. In another example, the syringes 72 are manufactured together with the injection ports 68. In one embodiment, the injection ports are manufactured independently of the bolus interconnectors. In another embodiment, the injection ports are manufactured integrally with the bolus interconnectors so that they extend therethrough.

At step 922, the sacrificial shell is removed.

It will be appreciated that the 3D printing process described in this disclosure may be selected from one of more types of 3D printing process including but not limited to Fused Deposition Modeling (FDM), Stereolithography (SLA), Masked Stereolithography (MSLA), Digital Light Processing (DLP), Selective Laser Sintering (SLS), Direct Metal Laser Sintering (DMLS), Selective Laser Melting (SLM), Electron Beam Melting (EBM), Multi Jet Fusion (MJF), Material Jetting (MJ), Drop on Demand (DOD), and Binder Jetting.

In another embodiment, the entire bolus is manufactured additively by, for example, 3D printing from two different materials. That is, both the internal relatively rigid endoskeleton structure and the surrounding relatively non-rigid skin interfacing layer are 3D printed sequentially or simultaneously. In this case the non-rigid skin interfacing layer is formed from a quick curing material such as polyurethane, silicone or Superflab.

It will be appreciated that the entire bolus may also be manufactured using an extrusion method, injection moulding process, subtractive manufacturing including milling and carving, or any other suitable manufacturing process, from two different materials. That is, both the internal relatively rigid endoskeleton structure and the surrounding relatively non-rigid skin interfacing layer are manufactured using an extrusion method, injection moulding process, subtractive manufacturing including milling and carving, or any other suitable manufacturing process, from two different materials.

It will also be appreciated that the manufacturing process for the entire bolus may be selected from two or more processes described above. In one example, the entire bolus is first 3D printed with different materials and then milled to achieve dimensional accuracy in accordance with the patient specific data. In another example, the internal relatively rigid endoskeleton structure is first manufactured using subtractive manufacturing (e.g. milling or carving) and then be covered with a relatively non-rigid skin interfacing material such as polyurethane, silicone, thermoplastic-waxes, latex rubbers or Superflab. The relatively non-rigid skin interfacing material can in turn be shaped using subtractive manufacturing (e.g. milling or carving) in according with the patient specific data.

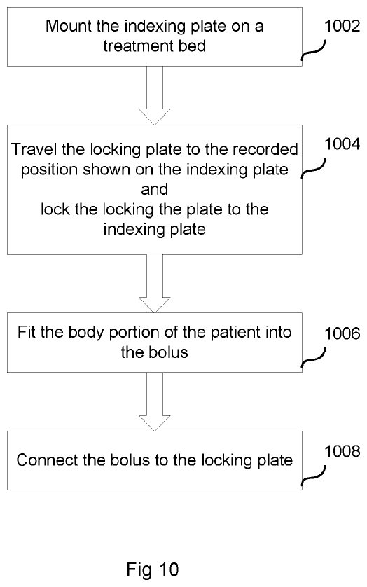

FIG. 10 illustrates a flow diagram of the steps involved in operating the bolus assembly. At step 1002, the indexing plate is mounted on a fixture (e.g. a fixed treatment bed). At step 1004, the locking plate travels within the series of locating tracks or channels on the indexing plate to the recorded position, and is then locked to the indexing plate by, for example, depressing the locking levers on the locking plate. The recorded position on the indexing plate may be viewable via the viewing window. At step 1006, the completed bolus may be fitted snugly to the body portion of the patient in exactly the right location. The bolus is then connected to the locking plate at step 1008. For example, the bolus connectors are received by the connecting assembly on the locking plate. Consequently, the bolus fitted to the body portion of the patient is mounted to the indexing plate using the indexing co-ordinates that were recorded for the C-channel so that the limb is in the correct position for radiation therapy. The order of the steps 1004, 1006 and 1008 may be interchangeable.

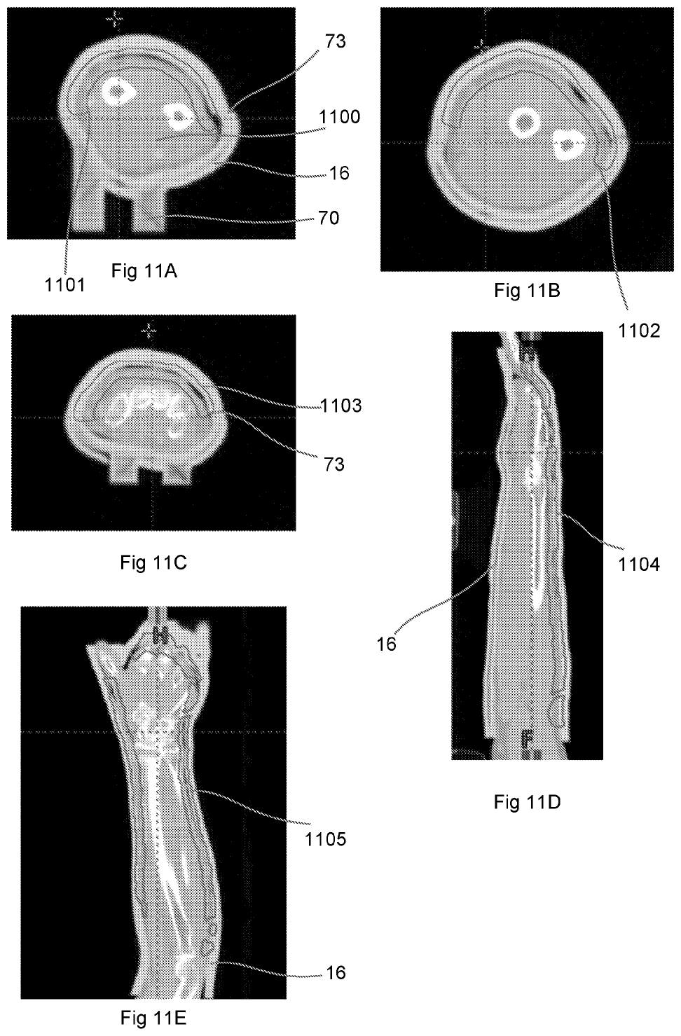

FIGS. 11A-11E illustrate an example of CT scanning results of a patient's right forearm 1100 positioned in a customised bolus component 16 manufactured according to the steps described in FIG. 9. In particular, FIG. 11A is an axial view of the middle right forearm. FIG. 11B is an axial view of the proximal right forearm. FIG. 1C is an axial view of the distal right forearm. FIG. 11D is a sagittal view of the right forearm. FIG. 11E is a coronal view of the right forearm. As illustrated, the bolus 16 is snugly and accurately fitted in place around the right forearm of the patient with a gap between the patient skin and bolus less than 2 mm (illustrated as 1101-1105 in FIGS. 11A-11E, respectively). As also can be seen from FIGS. 11A-11E, the bolus 16 and the patient limb 1100 have similarities in density of material indicating that the use of the bolus enables the delivery of the radiotherapy to the surface of the skin as required for treatment.

Evidence from testing the disclosed bolus components (based on both visual inspection and CT scan assessment) demonstrates that the disclosed bolus components are of robust construction and durable enough to survive the duration and rigour of typical routine clinical use. The disclosed bolus components also exhibit appropriate physical (e.g. physical density), transmission (e.g. of radiation dose) and uniformity properties for use in repeated sessions of radiotherapy treatment.

Clinical evidence from testing the disclosed bolus assemblies demonstrates that use of the disclosed bolus assemblies may facilitate the appropriate dose administration of radiation therapy, does not directly harm the user, nor exacerbate indirect radiation dermatitis. In addition, the disclosed assemblies may provide consistent user positioning between treatments and facilitate homogenous dose delivery across all users.

Now that arrangements of the present disclosure are described, it should be apparent to the skilled person in the art that at least one of the described arrangements may have one or more of the following advantages:
providing stabilisation to ensure minimal patient movement during both scanning and treatment;
providing comfort to the patient wearing the bolus without compromising stabilisation;
facilitating the process of positioning the bolus after scanning with the cradle in accordance with the scanned data.
enabling the delivery of the radiotherapy to the surface of the skin as required for treatment; and
providing a process of facilitating accurate manufacturing of the bolus and bolus positioning system.

It will be understood that the invention disclosed and defined in this specification extends to all alternative combinations of two or more of the individual features mentioned or evident from the text or drawings. All of these different combinations constitute various alternative aspects of the invention.

1. A bolus assembly for facilitating radiotherapy on a target body portion of a user, the bolus assembly comprising:
a customised bolus for fitting over the target body portion, the customised bolus comprising an internal relatively rigid endoskeleton structure and a surrounding relatively non-rigid skin interfacing layer;
an indexing plate mountable to a fixture and including a plurality of multiposition-enabling formations and corresponding reference locations;
at least one locking mechanism movable between and lockable relative to the multiposition-enabling formations;
at least one interconnecting formation for rigidly interconnecting the at least one locking mechanism and the customised bolus in a recordable position for enabling repeated radiotherapy treatments on the same target body portion of the user in the same position.

2. The bolus assembly of claim 1 wherein the multiposition-enabling formations comprise a plurality of tracks or channels within which the at least one locking mechanism is movable.

3. The bolus assembly of claim 2 wherein the at least one locking mechanism includes a locking plate and at least one clamping assembly movable between a clamping or locking position in which it is immobilised relative to the tracks or channels and a released position in which it is free to move along the tracks or channels.

4. The bolus assembly of claim 3 wherein in the released position the locking mechanism is free to move both linearly along the tracks or channels and to rotate angularly relative to the tracks or channels to facilitate positioning of the bolus prior to locking.

5. The bolus assembly of claim 3 wherein the recordable position is viewable through a viewing window provided on the locking plate.

6. The bolus assembly of claim 6 wherein the viewing window is configured as a through-hole to receive a locking or aligning pin for aligning the locking plate with a corresponding indexing recess in the indexing plate.

7. The bolus assembly of claim 1 wherein the at least one interconnecting formation includes a bolus connector extending from the endoskeleton structure, a complemental connector defined in the locking mechanism, and a lock formation for rigidly interlocking the bolus and complemental connector.

8. The bolus assembly of claim 1 wherein the reference locations have associated indexing indicia for enabling the position of the customised bolus to be recorded for a particular user.

9. The bolus assembly of claim 1 which includes a cradle component for initially locating a target body portion of the user, the cradle component being interchangeable with the customised bolus and being configured to locate and support a vacuum bag for immobilising the body portion prior to 3D scanning of the body portion.

10. The bolus assembly of claim 9 wherein the cradle component is radio translucent, and includes a cradle portion and at least one cradle connector extending from the cradle portion for interlocking with the complemental connector defined in the locking mechanism, and a lock formation for rigidly interlocking the cradle and complemental connector.

11. The bolus assembly of claim 1 wherein the relatively non-rigid skin interfacing layer of the customised bolus is provided with a longitudinally extending cut providing a clamshell-type opening to facilitate the bolus to be opened and snugly fitted in place around the target portion of the user.

12. The bolus assembly of claim 1, wherein the multiposition-enabling formations further comprise a plurality of locating pin holes for locating the at least one locking mechanism.

13. The bolus assembly of claim 12, wherein the at least one locking mechanism includes at least one locating pin for locating the at least one locking mechanism at a desired locating pin hole of the plurality of locating pin holes.

14. The bolus assembly of claim 8, wherein:
the at least one locking mechanism includes a locking pin extending through at least one aperture in the complemental connector;
the at least one interconnecting formation includes at least one aperture; and
in a locking state, the locking pin is inserted through aligned apertures of the complemental connector and the at least one interconnecting formation.

15. The bolus assembly of claim 1, wherein:
the at least one locking mechanism includes a cam lock; and
the at least one interconnecting formation includes at least one slot to receive the cam lock in a locking state.

16. A customised bolus for fitting over a target body portion of a user, the customised bolus comprising:
an internal relatively rigid endoskeleton structure;
a relatively non-rigid skin interfacing layer surrounding the internal relatively rigid endoskeleton structure; and
at least one connector extending from the endoskeleton structure, the connector being rigidly interconnectable to a locking mechanism which is detachably connectable to an indexing plate for enabling repeated radiotherapy treatments on the same target body portion of the user.

17. The customised bolus of claim 16 wherein the relatively non-rigid skin interfacing layer is overmoulded over the internal relatively rigid endoskeleton structure.

18. The customised bolus of claim 16 wherein the relatively non-rigid skin interfacing layer of the customised bolus is provided with a longitudinally extending cut providing a clamshell-type opening to facilitate the customised bolus to be opened and snugly fitted in place around the target portion of the user.

19. The customised bolus of claim 16 wherein the endoskeleton structure is formed with one or more reinforcing ribs.

20. A locking assembly for locking a cradle component for initially locating a target body portion of the user during 3D scanning or a bolus during radiotherapy on the target body portion of the user, the locking assembly comprising:
an indexing plate mountable to a fixture and including a plurality of multiposition-enabling formations and corresponding reference locations;
at least one locking mechanism movable between and lockable relative to the multiposition-enabling formations; and
at least one interconnecting formation for rigidly interconnecting the at least one locking mechanism and the bolus in a recordable position for enabling repeated radiotherapy treatments on the same target body portion of the user in the same position.

21. The locking assembly of claim 20 wherein the multiposition-enabling formations comprise a plurality of tracks or channels within which the at least one locking mechanism is movable.

22. The locking assembly of claim 21 wherein the at least one locking mechanism includes a locking plate and at least one clamping assembly movable between a clamping or locking position in which it is immobilised relative to the tracks or channels and a released position in which it is free to move along the tracks or channels.

23. The locking assembly of claim 22 wherein in the released position the locking mechanism is free to move both linearly along the tracks or channels and to rotate angularly relative to the tracks or channels to facilitate positioning of the bolus prior to locking.

24. The locking assembly of claim 22 wherein the recordable position is viewable through a viewing window provided on the locking plate.

25. The locking assembly of claim 24 wherein the viewing window is configured as a through-hole to receive a locking or aligning pin for aligning the locking plate with a corresponding indexing recess in the indexing plate.

26. The locking assembly of claim 20 wherein the at least one interconnecting formation includes a connector defined in the locking mechanism, and a lock formation for rigidly interlocking the bolus and connector.

27. The locking assembly of claim 20 wherein the reference locations have associated indexing indicia for enabling the position of the bolus to be recorded for a particular user.

28. The locking assembly of claim 20, wherein the multiposition-enabling formations further comprise a plurality of locating pin holes for locating the at least one locking mechanism.

29. The locking assembly of claim 28, wherein the at least one locking mechanism includes at least one locating pin for locating the at least one locking mechanism at a desired locating pin hole of the plurality of locating pin holes.

30. The locking assembly of claim 20, wherein:
the at least one locking mechanism includes a cam lock; and
the at least one interconnecting formation includes at least one slot to receive the cam lock in a locking state.
